(19) United States
(12) Patent Application Publication
(10) Pub. No.: US 2025/0212743 A1
(43) Pub. Date: Jul. 3, 2025

(54) LETTUCE PLANTS HAVING RESISTANCE TO DOWNY MILDEW

(71) Applicant: Seminis Vegetable Seeds, Inc., St. Louis, MO (US)

(72) Inventors: Eleni Bachlava, Fairfield, CA (US); Andrea K. Knox, Sacramento, CA (US); Gregory Stuart Pape, San Juan Bautista, CA (US)

(21) Appl. No.: 18/999,130

(22) Filed: Dec. 23, 2024

Related U.S. Application Data
(60) Provisional application No. 63/616,273, filed on Dec. 29, 2023.

(51) Int. Cl. A01H 1/04; C12Q 1/6895

(52) U.S. Cl. CPC A01H 1/045; C12Q 1/6895; C12Q 2600/13; C12Q 2600/156

(57) ABSTRACT

Lettuce (Lactuca sativa) plants exhibiting resistance to downy mildew disease caused by Bremia lactucae are provided, together with methods of producing, identifying, or selecting plants or germplasm with a downy mildew resistance phenotype. Such plants include lettuce plants comprising introgressed genomic regions conferring pest resistance. Compositions, including novel polymorphic markers for detecting plants comprising introgressed loci, are further provided.

### CROSS-REFERENCE TO RELATED APPLICATIONS

This application claims priority to U.S. Provisional Patent Application No. 63/616,273, filed Dec. 29, 2023, the entire disclosure of which is incorporated herein by reference.

### INCORPORATION OF SEQUENCE LISTING

A sequence listing containing the file named “SEMB055US_ST26.xml” which is 11 kilobytes (measured in MS-Windows®) and created on Dec. 13, 2024, and comprises 7 sequences, is incorporated herein by reference in its entirety.

### FIELD

The present disclosure relates to methods and compositions for producing lettuce plants exhibiting increased resistance to downy mildew disease caused by Bremia lactucae.

### BACKGROUND

Host plant resistance is an important trait in agriculture, particularly in the area of food crop production. Bremia lactucae, is the causal agent of Downy Mildew (DM) in lettuce. This pathogen represents a major threat for lettuce production around the world, as it generates lesions in foliar tissues, reducing the quality and marketability of lettuce. Furthermore, Bremia lactucae is highly variable and dynamic making DM resistance a key trait for growers isolates changes rapidly. Although some DM resistance alleles have been identified, the mapping and introduction of sustainable resistance to viruses remains one of the main challenges of modern plant breeding, especially in vegetables. Moreover, the globalization of food supply chains favors the spread of new virus strains or species. Therefore, a continuing need exists in the art to identify new resistance alleles conferring increased resistance to DM as well as more effective methods of introducing those resistance alleles into commercial lines to provide new varieties with improved resistance to Bremia lactucae.

### SUMMARY

In one aspect, provided herein is an elite Lactuca sativa plant comprising a recombinant chromosomal segment from Lactuca serriola on chromosome 9, wherein said chromosomal segment is flanked in the genome of said plant by marker locus M1 (SEQ ID NO: 1) and marker locus M2 (SEQ ID NO: 2), and wherein said chromosomal segment comprises an allele conferring resistance to Bremia lactucae relative to a plant lacking said chromosomal segment. In some embodiments, said chromosomal segment is flanked in the genome of said plant by marker locus M1 (SEQ ID NO: 1) and marker locus M6 (SEQ ID NO: 6). In other embodiments, said chromosomal segment comprises a marker locus selected from the group consisting of marker locus M5 (SEQ ID NO: 5) and marker locus M6 (SEQ ID NO: 6) on chromosome 9. In some embodiments, said chromosomal segment comprises marker locus M5 (SEQ ID NO: 5) on chromosome 9. In still other embodiments, the plant is homozygous for said chromosomal segment. In certain embodiments, a representative sample of seed comprising said allele has been deposited under NCMA Accession No. 202212295, NCMA Accession No. 202307003, or NCMA Accession No. 202306045. In further embodiments, said chromosomal segment confers resistance to at least Bremia lactucae races Bl: 17-28EU; confers resistance to at least Bremia lactucae races Bl: 29-37EU; confers resistance to at least Bremia lactucae races Bl: 5-9US; confers resistance to at least Bremia lactucae races Bl: 38EU and Bl: 39EU; confers resistance to at least Bremia lactucae races Bl: 40EU and Bl: 41EU; and/or confers resistance to at least Bremia lactucae races Bl: 38-41EU. In some embodiments, said plant comprises a marker locus associated with said chromosomal segment selected from the group consisting of marker locus M3 (SEQ ID NO: 3) and marker locus M4 (SEQ ID NO: 4). In further embodiments, said plant comprises said plant comprises: a marker locus selected from the group consisting of marker locus M3 (SEQ ID NO: 3), marker locus M4 (SEQ ID NO: 4), marker locus M5 (SEQ ID NO: 5), and marker locus M6 (SEQ ID NO: 6); marker locus M3 (SEQ ID NO: 3), M4 (SEQ ID NO: 4), and M6 (SEQ ID NO: 6); marker locus M5 (SEQ ID NO: 5), and M6 (SEQ ID NO: 6); marker locus M3 (SEQ ID NO: 3), and M6 (SEQ ID NO: 6); or marker locus M5 (SEQ ID NO: 5).

In another aspect, cells, seed, and plant parts comprising a recombinant chromosomal segment from Lactuca serriola on chromosome 9, wherein said chromosomal segment is flanked in the genome of said plant by marker locus M1 (SEQ ID NO: 1) and marker locus M2 (SEQ ID NO: 2), and wherein said chromosomal segment comprises an allele conferring resistance to Bremia lactucae relative to a plant lacking said chromosomal segment are provided. Cells, seeds, and plant parts comprising said chromosomal segment are further provided.

In a further aspect, a recombinant DNA segment (which may be referred to as a nucleic acid molecule) comprising a Bremia lactucae resistance allele from Lactuca serriola, wherein said DNA segment comprises the sequence of marker locus M1 (SEQ ID NO: 1) and marker locus M2 (SEQ ID NO: 2) is provided. In some embodiments, said recombinant DNA segment comprises a marker locus sequence selected from the group consisting of marker locus M3 (SEQ ID NO: 3), marker locus M4 (SEQ ID NO: 4), marker locus M5 (SEQ ID NO: 5), and marker locus M6 (SEQ ID NO: 6). In certain embodiments, said recombinant DNA segment further comprises: marker locus M3 (SEQ ID NO: 3), M4 (SEQ ID NO: 4), and M6 (SEQ ID NO: 6); marker locus M5 (SEQ ID NO: 5) and M6 (SEQ ID NO: 6); marker locus M3 (SEQ ID NO: 3), and M6 (SEQ ID NO: 6); or marker locus M5 (SEQ ID NO: 5). In some embodiments, a representative sample of seed comprising said recombinant DNA segment has been deposited under NCMA Accession No. 202212295, NCMA Accession No. 202307003, or NCMA Accession No. 202306045. In further embodiments, said recombinant DNA segment is further defined as comprised within a plant, plant part, plant cell, or seed.

In another aspect, methods are provided for producing an elite Lactuca sativa plant with resistance to Bremia lactucae comprising introgressing into said plant a Bremia lactucae resistance allele from Lactuca serriola within a recombinant chromosomal segment flanked in the genome of said plant by marker locus M1 (SEQ ID NO: 1) and marker locus M2 (SEQ ID NO: 2) on chromosome 9; wherein said Bremia lactucae resistance allele confers to said plant resistance to Bremia lactucae relative to a plant lacking said allele, and wherein said introgressing comprises marker-assisted selection. In further embodiments, said introgressing comprises: crossing a plant comprising said recombinant chromosomal segment with itself or with a second Lactuca sativa plant of a different genotype to produce one or more progeny plants; and selecting a progeny plant comprising said recombinant chromosomal segment. In yet further embodiments, selecting the progeny plant is an F₂-F₆ progeny plant. In certain embodiments, selecting a progeny plant comprises detecting nucleic acids comprising marker locus M1 (SEQ ID NO: 1), marker locus M2 (SEQ ID NO: 2), marker locus M3 (SEQ ID NO: 3), marker locus M4 (SEQ ID NO: 4), marker locus M5 (SEQ ID NO: 5), or marker locus M6 (SEQ ID NO: 6). In still further embodiments, said introgressing further comprises backcrossing or assaying for said resistance to Bremia lactucae. Lettuce plants obtainable by the methods disclosed herein are further provided.

In further aspects, methods are provided for obtaining a seed of a Lactuca sativa plant exhibiting resistance to Bremia lactucae, comprising: crossing a Lactuca sativa plant comprising a recombinant chromosomal segment from Lactuca serriola on chromosome 9, wherein said chromosomal segment is flanked in the genome of said plant by marker locus M1 (SEQ ID NO: 1) and marker locus M2 (SEQ ID NO: 2), and wherein said chromosomal segment comprises an allele conferring resistance to Bremia lactucae relative to a plant lacking said chromosomal segment, with itself or with a second Lactuca sativa plant of a different genotype; and obtaining a seed resulting from said crossing that comprises said recombinant chromosomal segment. In certain embodiments, obtaining said seed comprises detecting a marker locus genetically linked to said chromosomal segment. In other embodiments, said seed when grown produces an F₂-F₆ progeny plant. In specific embodiments, said crossing comprises backcrossing. In yet further embodiments, obtaining the seed comprises detecting nucleic acids comprising marker locus M1 (SEQ ID NO: 1), marker locus M2 (SEQ ID NO: 2), marker locus M3 (SEQ ID NO: 3), marker locus M4 (SEQ ID NO: 4), marker locus M5 (SEQ ID NO: 5), or marker locus M6 (SEQ ID NO: 6). Lettuce plants obtainable by the methods disclosed herein are further provided.

In yet another aspect, methods are provided for identifying a Lactuca sativa plant exhibiting resistance to Bremia lactucae, comprising: obtaining a population of progeny plants having a parent comprising resistance to Bremia lactucae; screening said population with at least one nucleic acid marker to detect a polymorphism genetically linked to Bremia lactucae resistance; and identifying in said population at least a first progeny plant comprising a haplotype associated with Bremia lactucae resistance, wherein the haplotype comprises a Bremia lactucae resistance allele from Lactuca serriola at a locus flanked in the genome of said plant by marker locus M1 (SEQ ID NO:1) and marker locus M2 (SEQ ID NO:2) on chromosome 9. Alternatively, methods are provided for identifying a Lactuca sativa plant exhibiting resistance to Bremia lactucae wherein a population of progeny plants having a parent comprising resistance to Bremia lactucae are obtained; and wherein the method further comprises screening said population with at least one nucleic acid marker to detect a polymorphism genetically linked to Bremia lactucae resistance; and identifying in said population at least a first progeny plant comprising a haplotype associated with Bremia lactucae resistance, wherein the haplotype comprises a Bremia lactucae resistance allele from Lactuca serriola at a locus flanked in the genome of said plant by marker locus M1 (SEQ ID NO:1) and marker locus M2 (SEQ ID NO:2) on chromosome 9. In certain embodiments, identifying said progeny plant comprises: detecting a marker locus within or genetically linked to a chromosomal segment flanked in the genome of said plant by marker locus M1 (SEQ ID NO: 1) and marker locus M2 (SEQ ID NO: 2) on chromosome 9; detecting at least one polymorphism at a locus selected from the group consisting of M1 (SEQ ID NO: 1), marker locus M2 (SEQ ID NO: 2), marker locus M3 (SEQ ID NO: 3), marker locus M4 (SEQ ID NO: 4), marker locus M5 (SEQ ID NO: 5), and marker locus M6 (SEQ ID NO: 6); or detecting at least one polymorphism at marker locus M5 (SEQ ID NO: 5). In some embodiments, the haplotype comprises a Bremia lactucae resistance allele from Lactuca serriola at a locus flanked in the genome of said plant by marker locus M1 (SEQ ID NO:1) and marker locus M6 (SEQ ID NO:6) on chromosome 9. In further embodiments, said progeny plant is an F₂-F₆ progeny plant, or producing said progeny plant comprises backcrossing. In still further embodiments, screening said population comprises PCR, single strand conformational polymorphism analysis, denaturing gradient gel electrophoresis, cleavage fragment length polymorphism analysis, TAQMAN assay, and/or DNA sequencing. In specific embodiments, a representative sample of seed comprising said Bremia lactucae resistance allele has been deposited under NCMA Accession No. 202212295, NCMA Accession No. 202307003, or NCMA Accession No. 202306045.

In another aspect, methods are provided for identifying a lettuce plant comprising a Bremia lactucae resistance allele from Lactuca serriola, comprising: obtaining nucleic acids from at least a first lettuce plant; and identifying in said nucleic acids the presence of at least a first genetic marker indicative of the presence of a chromosomal segment flanked in the genome of said plant by marker locus M1 (SEQ ID NO:1) and marker locus M2 (SEQ ID NO:2) on chromosome 9; wherein said chromosomal segment comprises a Bremia lactucae resistance allele conferring to said plant increased resistance to Bremia lactucae compared to a plant not comprising said allele. In some embodiments, said identifying comprises detecting a marker genetically linked to M1 (SEQ ID NO: 1), marker locus M2 (SEQ ID NO: 2), marker locus M3 (SEQ ID NO: 3), marker locus M4 (SEQ ID NO: 4), marker locus M5 (SEQ ID NO: 5), or marker locus M6 (SEQ ID NO: 6). In other embodiments, the first genetic marker is indicative of the presence of a chromosomal segment flanked in the genome of said plant by marker locus M1 (SEQ ID NO:1) and marker locus M6 (SEQ ID NO:6) on chromosome 9.

In yet another aspect, methods are provided for selecting a lettuce plant or lettuce seed, said method comprising: detecting in a population of lettuce plants or lettuce seeds a lettuce plant or lettuce seed comprising an allele from Lactuca serriola that confers to said plant resistance to Bremia lactucae compared to a plant not comprising said allele; and selecting said lettuce plant or lettuce seed comprising said allele from Lactuca serriola that confers to said plant a resistance to Bremia lactucae, wherein said allele is located on chromosome 9. In some embodiments, said allele is linked to any marker selected from the group consisting of marker M1 (SEQ ID NO: 1), marker locus M2 (SEQ ID NO: 2), marker locus M3 (SEQ ID NO: 3), marker locus M4 (SEQ ID NO: 4), marker locus M5 (SEQ ID NO: 5), or marker locus M6 (SEQ ID NO: 6) on chromosome 9. In other embodiments, said plant is homozygous for said allele from Lactuca serriola; or is heterozygous for said allele from Lactuca serriola. In further embodiments, said allele is linked to marker locus M3 (SEQ ID NO: 3), M4 (SEQ ID NO: 4), and M6 (SEQ ID NO: 6); marker locus M5 (SEQ ID NO: 5) and M6 (SEQ ID NO: 6); marker locus M3 (SEQ ID NO: 3) and M6 (SEQ ID NO: 6); or marker locus M5 (SEQ ID NO: 5). In still further embodiments, marker locus M3 comprises a C at position 106 of SEQ ID NO:3; marker locus M4 comprises an A at position 61 of SEQ ID NO:4; marker locus M5 comprises an A at position 301 of SEQ ID NO:5; or marker locus M6 comprises a C at position 101 of SEQ ID NO:6. In still further embodiments, said lettuce plant or lettuce seed is an elite plant or a seed of an elite plant. Plants identifiable by the methods described herein are also provided.

In further aspects, the present disclosure provides a nucleic acid molecule comprising an allele conferring a Bremia lactucae resistance to a lettuce plant, wherein the nucleic acid molecule is obtainable or can be obtained from seed deposited under NCMA Accession No. 202212295, NCMA Accession No. 202307003, or NCMA Accession No. 202306045, and wherein the nucleic acid molecule comprises at least one marker selected from the group consisting of marker locus M3 (SEQ ID NO: 3), marker locus M4 (SEQ ID NO: 4), marker locus M5 (SEQ ID NO: 5), and marker locus M6 (SEQ ID NO: 6). Optionally, the nucleic acid molecule comprising an allele conferring a Bremia lactucae resistance to a lettuce plant can be isolated. In further embodiments, the nucleic acid molecule may further comprise: a) marker locus M3 (SEQ ID NO: 3), M4 (SEQ ID NO: 4), and M6 (SEQ ID NO: 6); b) marker locus M5 (SEQ ID NO: 5) and M6 (SEQ ID NO: 6); c) marker locus M3 (SEQ ID NO: 3) and M6 (SEQ ID NO: 6); or d) marker locus M5 (SEQ ID NO: 5). In still further embodiments, SEQ ID NO:3 comprises a C at position 106; SEQ ID NO: 4 comprises an A at position 61; SEQ ID NO:5 comprises an A at position 301, and SEQ ID NO: 6 comprises a C at position 101. In specific embodiments, the nucleic acid molecule may further comprise at least one marker locus comprising: SEQ ID NO:3 comprising a C at position 106; SEQ ID NO:4 comprising an A at position 61; SEQ ID NO:5 comprising an A at position 301; or SEQ ID NO:6 comprising a C at position 101. In some embodiments, the nucleic acid molecule further comprises at least one marker selected from the group consisting of marker M1 (SEQ ID NO: 1) and marker locus M2 (SEQ ID NO: 2). In other embodiments, the nucleic acid molecule may further comprise at least one marker locus comprising: SEQ ID NO:1 comprising an A at position 61; SEQ ID NO: 1 comprising a G at position 61; SEQ ID NO:2 comprising an A at position 61; or SEQ ID NO:2 comprising a G at position 61.

### BRIEF DESCRIPTION OF THE DRAWINGS

FIG. 1: Shows an overview of the marker positions of marker locus M1 and marker locus M2 and chromosomal region on chromosome 9 comprising the resistance alleles that confer resistance to Bremia lactucae in lettuce.

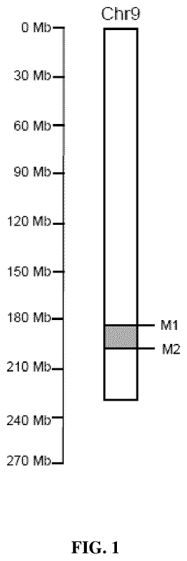

### DETAILED DESCRIPTION

Lettuce (L. sativa) is an important vegetable crop that is produced and consumed on a global scale. The most important disease that affects the quality of lettuce crops is downy mildew, caused by the oomycete Bremia lactucae. To control B. lactucae infection in lettuce, breeders typically employ a strategy that combines the use of fungicides with lettuce varieties that have genetic resistance to B. lactucae. However, fungicide use is expensive and heavy use can lead to the development of B. lactucae races that are no longer susceptible to fungicides. In addition, increasingly restrictive regulations over fungicide use along with a consumer push for reduced pesticide use limits the options for B. lactucae control measures by lettuce growers. These factors have led growers to rely more on the genetic resistance of the lettuce varieties they grow. However, pathogen evolution has reduced the efficacy of the resistance genes typically deployed to protect lettuce crops. The International Bremia Evaluation Board (IBEB) is responsible for officially nominating and naming the races of Bremia lactucae. In Europe, to date isolates Bl: 1-41EU have been named and of those, isolates Bl: 29-41EU are currently identified as commercially relevant. In the United States, to date isolates Bl: 1-9US have been named and of those, isolates Bl: 5-9US are currently identified as commercially relevant. However, novel races are regularly identified. It is a priority for lettuce breeders to develop lettuce varieties having resistance to all commercially relevant known B. lactucae races, and other additional relevant races.

More than 50 genes that confer resistance to B. lactucae infection have been identified in L. sativa and its wild relatives L. serriola and L. saligna. Breeders have been able to utilize the genes from L. serriola and L. saligna in the production of resistant lettuce varieties due to the fact that L. serriola and L. saligna are closely related to L. sativa. Several B. lactucae resistance genes have also been identified in the wild relative L. virosa, but introgression of genes from L. virosa into L. sativa is challenging due to lingering fertility barriers. The known resistance genes from L. serriola can be mapped to two gene clusters in the lettuce genome. One cluster is located on chromosome 2 while the other cluster is located on chromosome 4. While breeders appear to have multiple resistance genes to choose from, the location of the genes in such gene clusters and the proximity between the genes has greatly limited the ability to stack available resistance genes.

After a new B. lactucae isolate is determined to be commercially relevant, a lettuce breeder typically must identify a resistance gene that confers resistance to the new isolate and incorporate this additional resistance locus along with the loci conferring resistance to all previously known isolates into a relevant lettuce variety. One method to identify genes that confer resistance to new B. lactucae isolates is to evaluate known resistance genes to determine if the known gene confers resistance to one or more of the new isolate(s). After a source of resistance to the new isolate(s) is identified, the resistance locus must be incorporated into the relevant lettuce germplasm for use in the breeding program to develop new lettuce varieties. If a known gene confers resistance to the new isolate, it is easy for the breeders to incorporate the resistance locus into the breeding program. However, if a new resistance allele is required to confer resistance, the new allele must be integrated into varieties that likely already include other loci that confer resistance to other B. lactucae races. Such new allele must be introgressed along with any other relevant loci so as to ensure that the resulting plants or varieties exhibit resistance to the relevant B. lactucae races or isolates officially named by IBEB, which pose a threat to the lettuce industry in the relevant commercial region. Although multiple B. lactucae resistance genes have been previously combined in a single lettuce variety, the combination of these genes did not result in a resistance profile that provided resistance to all known officially named B. lactucae races. Another method used to identify genes that confer resistance to new B. lactucae isolates is through screening of wild lettuce accessions; however, such methods are unpredictable.

63 QTLs for polygenic resistance or qDMR (QTLs for lettuce downy mildew resistance) have previously been identified randomly distributed across all nine chromosomes of Lactuca sativa cv. Salinas (Simko et al, 2022). In particular, five qDMRs distributed along chromosome 9 have been identified, including qDMR9.1, qDMR9.2, qDMR9.3 (Parra et al., 2020); and qDMR9.4, and qDMR9.5 (Simko et al, 2022). However, the resistance alleles disclosed herein are distinct from those known in the art. Moreover, previously described qDMR9.1 and qDMR9.2 originated from L. sativa, and qDMR9.3 originated from L. saligna.

The present disclosure represents a significant advance in that it provides, in one embodiment, novel alleles from Lactuca serriola on chromosome 9 which can be introgressed in a lettuce plant to confer resistance to B. lactucae, as well as methods for the production thereof. Such plants can be referred to as plants of B. lactucae resistant lettuce varieties. Methods of producing such B. lactucae resistant lettuce plants, lines, and varieties are further provided. Also disclosed herein are molecular markers that can be used to introgress and track the B. lactucae resistance. The inventors surprisingly identified multiple Lactuca serriola sources showing resistance to Bremia lactucae European isolates Bl: 1-24 EU. Further testing showed that the alleles were resistant to all of the relevant European isolates identified at the time, including Bl: 17-37EU. It is noted that isolates 1-16 were not commercially relevant in Europe at the time and isolates 17-28 are currently no longer commercially relevant in Europe. The alleles also exhibited resistance for the relevant U.S. isolates including Bl: 5-9US. It is noted that isolates 1˜4 are no longer commercially relevant in the U.S. Moreover, the allele providing resistance from each of the respective sources was located in a region of the genome flanked by marker locus M1 (SEQ ID NO: 1) and marker locus M2 (SEQ ID NO: 2). The resistance profile and alleles are distinct from those known in the art. In addition, markers which can be used to introgress and track the novel alleles are provided herein, allowing the alleles to be introgressed and tracked during development of new varieties. Furthermore, through use of such markers and the methods described herein, one of skill in the art may provide resistance to additional isolates, increase the level of B. lactucae resistance exhibited by a lettuce plant, and identify plants exhibiting an increased level of B. lactucae resistance conferred by a novel allele from Lactuca serriola on chromosome 9.

In some embodiments, the novel B. lactucae resistance allele may be defined as located within a recombinant chromosomal segment from Lactuca serriola flanked by marker locus M1 (SEQ ID NO: 1) and marker locus M2 (SEQ ID NO: 2) on chromosome 9. Such a chromosomal segment can comprise one or more of marker locus M5 (SEQ ID NO: 5) and marker locus M6 (SEQ ID NO: 6). Marker locus M1 comprises a [A/G] change at 181,160,842 bp of the public L. sativa reference genome Lsat_Salinas_v8, marker locus M2 comprises a [A/G] change at 195,030,172 bp of the public L. sativa reference genome Lsat_Salinas_v8. Markers M5 and M6 are interstitial markers, where marker locus M5 is a SNP marker with a [T/A] change at 188,962,368 bp of the public L. sativa reference genome Lsat_Salinas_v8, and marker locus M6 is a SNP marker with a [T/C] change at 192,658,443 bp of the public L. sativa reference genome Lsat_Salinas_v8. In other embodiments, the chromosomal segment may be defined as located within a recombinant chromosomal segment from Lactuca serriola flanked by marker locus M1 (SEQ ID NO: 1) and marker locus M6 (SEQ ID NO: 6); or marker locus M3 (SEQ ID NO: 3) and marker locus M6 (SEQ ID NO: 6). Marker locus M3 is a SNP marker with a [T/C] change at 176,238,419 bp of the public L. sativa reference genome Lsat_Salinas_v8. The public genome of lettuce is available at, for example lgr.genomecenter.ucdavis.edu, and one skilled in the art would understand how to locate the marker sequences provided for the first time in the instant application on any version (or later version) of the public genome.

In other embodiments, provided herein are plants comprising a recombinant chromosomal segment from Lactuca serriola on chromosome 9, wherein said chromosomal segment comprises an allele conferring resistance to Bremia lactucae relative to a plant lacking said chromosomal segment, and wherein said plant comprises a marker locus genetically linked to said chromosomal segment selected from the group consisting of marker locus M3 (SEQ ID NO: 3), marker locus M4 (SEQ ID NO: 4). Marker locus M4 is a SNP marker with a [G/A] change at 177,199,102 bp of the public L. sativa reference genome Lsat_Salinas_v8. In specific embodiments, said plants can further comprise marker locus M7. Marker locus M7 is a SNP maker with a [T/C] change at 198,494,489 bp of the public L. sativa reference genome Lsat_Salinas_v8. The public genome of lettuce is available at, for example lgr.genomecenter.ucdavis.edu, and one skilled in the art would understand how to locate the marker sequences provided for the first time in the instant application on any version (or later version) of the public genome.

In another aspect, provided herein is a plant comprising a recombinant chromosomal segment from Lactuca serriola on chromosome 9, wherein said chromosomal segment is flanked in the genome of said plant by marker locus M1 (SEQ ID NO: 1) and marker locus M2 (SEQ ID NO: 2), and wherein said plant can comprise one or more of marker locus M3 (SEQ ID NO: 3), marker locus M4 (SEQ ID NO: 4), marker locus M5 (SEQ ID NO: 5), and marker locus M6 (SEQ ID NO: 6). In particular embodiments, said plant can comprise marker locus M3 (SEQ ID NO: 3), and marker locus M6 (SEQ ID NO: 6); marker locus M5 (SEQ ID NO: 5), and marker locus M6 (SEQ ID NO: 6); or marker locus M3 (SEQ ID NO: 3), marker locus M4 (SEQ ID NO: 4), and marker locus M6 (SEQ ID NO: 6). In some embodiments, provided herein is a plant comprising a recombinant chromosomal segment from Lactuca serriola on chromosome 9, wherein said chromosomal segment is flanked in the genome of said plant by marker locus M1 (SEQ ID NO: 1) and marker locus M2 (SEQ ID NO: 2), and wherein said plant comprises marker locus M5 (SEQ ID NO: 5). In specific embodiments, said plant can further comprise marker locus M7 (SEQ ID NO: 7).

In yet another aspect, the Bremia lactucae resistance alleles from Lactuca serriola described herein may be defined as being present on chromosome 9 and conferring resistance to at least Bremia lactucae isolates Bl: 28-37EU. In yet another aspect, the Bremia lactucae resistance alleles from Lactuca serriola described herein may be defined as being present on chromosome 9 and conferring resistance to at least Bremia lactucae isolates Bl: 5-9US. In still another aspect, the Bremia lactucae resistance alleles from Lactuca serriola described herein may be defined as being present on chromosome 9 and conferring resistance to at least Bremia lactucae isolates Bl: 38EU and Bl: 39EU. In still yet another aspect, the Bremia lactucae resistance alleles from Lactuca serriola described herein may be defined as being present on chromosome 9 and conferring resistance to at least Bremia lactucae isolates Bl: 40EU and Bl: 40EU, or to at least Bremia lactucae isolates Bl: 38-41EU. Nucleotide sequences associated with and diagnostic for the resistance alleles are provided herein, e.g. SEQ ID NOs: 3-7. In other embodiments, resistance alleles providing said resistance may be defined as from, or sharing, a genetic source selected from accessions SVLD0093, LAC23B, and CHD-8W22-6051V, representative deposits of seed of which were made with the NCMA under accession numbers 202212295, 202307003, and 202306045, respectively.

In certain embodiments, the disclosure provides methods of producing or selecting a lettuce plant exhibiting resistance to B. lactucae comprising: a) crossing a lettuce plant provided herein with itself or with a second lettuce plant of a different genotype to produce one or more progeny plants; and b) selecting a progeny plant comprises detecting nucleic acids comprising marker locus M1 (SEQ ID NO: 1), marker locus M2 (SEQ ID NO: 2), marker locus M3 (SEQ ID NO: 3), marker locus M4 (SEQ ID NO: 4), marker locus M5 (SEQ ID NO: 5), or marker locus M6 (SEQ ID NO: 6).

Because genetically diverse plant lines can be difficult to cross, the introgression of B. lactucae resistance loci and/or alleles into cultivated lines using conventional breeding methods could require prohibitively large segregating populations for progeny screens with an uncertain outcome. Marker-assisted selection (MAS) is therefore essential for the effective introgression of loci that confer resistance B. lactucae into elite cultivars. For the first time, the present disclosure enables effective MAS by providing improved and validated markers for detecting genotypes associated with B. lactucae resistance without the need to grow large populations of plants to maturity in order to observe the phenotype.

In particular, genetic markers in linkage disequilibrium with the resistance alleles of the present disclosure may permit efficient introduction of DM-resistance into essentially any lettuce genome. This also results in significant economization by permitting substitution of costly, time-intensive, and potentially unreliable phenotypic assays. Further, breeding programs can be designed to explicitly drive the frequency of specific favorable phenotypes by targeting particular genotypes. Fidelity of these associations may be monitored continuously to ensure maintained predictive ability and, therefore, informed breeding decisions. In accordance with the disclosure, one of skill in the art may identify a candidate germplasm source possessing a desirable DM-resistant phenotype, such as from an accession described herein. One embodiment of the disclosure comprises using the materials and methods of the disclosure to obtain an allele conferring resistance to DM from any additional lettuce accessions.

In a further aspect, the disclosure provides a method of selecting a plant with resistance to Bremia lactucae comprising selecting said plant based on the presence in the genome of the plant at least a first genetic polymorphism in or in genetic linkage disequilibrium with a chromosomal segment conferring said resistance, wherein the locus is flanked in the genome by marker locus M1 (SEQ ID NO: 1) and marker locus M2 (SEQ ID NO: 2), marker locus M1 (SEQ ID NO: 1) and marker locus M6 (SEQ ID NO: 6), or sequences at least 95% identical thereto. In an embodiment, the plant is a Lactuca sativa plant comprising in its genome at least one recombinant chromosomal segment from Lactuca serriola. In another embodiment, the plant is a Lactuca serriola plant. In specific embodiments, the at least first genetic polymorphism in or in genetic linkage disequilibrium with a chromosomal segment conferring said resistance is selected from SEQ ID NOs: 3-6. In yet another aspect, provided herein is a method of selecting a plant with resistance to Bremia lactucae comprising detecting marker locus M3 (SEQ ID NO: 3), marker locus M4 (SEQ ID NO: 4), marker locus M5 (SEQ ID NO: 5), or marker locus M6 (SEQ ID NO: 6); or a marker locus genetically linked thereto. In yet another embodiment, the method further comprising the step of confirming the presence of said resistance with a phenotypic assay. In still another embodiment, the method further comprises crossing said plant comprising the locus with another lettuce plant to produce a progeny plant comprising said locus.

I. Genomic Regions, Loci, and Polymorphisms in Lettuce Associated with Resistance to Downy Mildew Disease

The disclosure provides novel recombinant chromosomal segments or alleles of one or more loci associated with resistance to downy mildew disease in lettuce, together with polymorphic nucleic acids and linked markers for tracking the chromosomal segments or alleles during plant breeding.

Lactuca sativa accessions exhibiting B. lactucae resistance are known in the art and may be used in accordance with certain embodiments of the disclosure. It may also be possible to use other lettuce types including L. serriola, L. virosa, and L. saligna. Lactuca sativa accession CGN05813 and Lactuca saligna accession CGN05271, which can be obtained from the Center for Genetic Resources (Wageningen, The Netherlands), also exhibit resistance to B. lactucae. Accessions for L. serriola lines exhibiting resistance to various B. lactucae races are given, for example, in Lebeda et al., Eur J Plant Pathol, 138:597-640, 2014. Other B. lactucae resistance sources have also been described and are known in the art (see, for example, Simko et al., Phytopathology 105 (9): 1220-1228, 2015; Van Hese et al., Eur J. Plant Pathology 144:431-441, 2016; and Parra et al., Euphytica 210:309-326, 2016). Lactuca accessions have been collected in numerous locales including France and Portugal and can be found in a number of germplasm banks including Center for Genetic Resources, the Netherlands (CGN) and National Plant Germplasm System (NPGS).

II. Introgression of Genomic Regions Associated with Resistance to Downy Mildew Disease Marker-assisted introgression involves the transfer of a chromosomal region or allele defined by one or more markers from a first genetic background to a second. Offspring of a cross that contain the introgressed genomic region can be identified by the combination of markers characteristic of the desired introgressed genomic region from a first genetic background and both linked and unlinked markers characteristic of the second genetic background.

The present disclosure provides novel accurate markers for identifying and tracking introgression of one or more of the genomic regions disclosed herein from a downy mildew resistant plant into a cultivated line. The disclosure further provides markers for identifying and tracking the novel introgressions disclosed herein during plant breeding, including the markers set forth in Table 2.

Markers within or linked to any of the genomic intervals of the present disclosure may be useful in a variety of breeding efforts that include introgression of genomic regions associated with pest resistance into a desired genetic background. For example, a marker within 40 cM, 20 cM, 15 cM, 10 cM, 5 CM, 2 cM, or 1 cM of a marker associated with pest resistance described herein can be used for marker-assisted introgression of genomic regions associated with a pest resistant phenotype.

Lettuce plants comprising one or more introgressed regions associated with a desired phenotype wherein at least 10%, 25%, 50%, 75%, 90%, or 99% of the remaining genomic sequences carry markers characteristic of the recurrent parent germplasm are also provided. Lettuce plants comprising an introgressed region comprising regions closely linked to or adjacent to the genomic regions and markers provided herein and associated with a pest resistance phenotype are also provided.

### III. Development of Lettuce Varieties Resistant to Downy Mildew Disease

For most breeding objectives, commercial breeders work with germplasm that is “cultivated,” “cultivated type,” or “elite”. This germplasm is easier to breed because it generally performs well when evaluated for horticultural performance. A number of cultivated lettuce types have been developed, including L. sativa, which is agronomically elite and appropriate for commercial cultivation. Lettuce cultivar groups include, but are not limited to, the Cos, Cutting, Stalk (or Asparagus), Butterhead, Crisphead (or Iceberg or Cabbage), Latin and Oilseed groups (De Vries, Gen. Resources and Crop Evol. 44:165-174, 1997). However, the performance advantage a cultivated germplasm provides can be offset by a lack of allelic diversity. Breeders generally accept this tradeoff because progress is faster when working with cultivated material than when breeding with genetically diverse sources.

In contrast, when cultivated germplasm is crossed with non-cultivated germplasm, a breeder can gain access to novel alleles from the non-cultivated type. However, this approach presents significant difficulties due to fertility problems associated with crosses between diverse lines, and negative linkage drag from the non-cultivated parent. In lettuce plants, non-cultivated types such as L. serriola can provide alleles associated with disease resistance. However, these non-cultivated types may have poor horticultural qualities.

The process of introgressing desirable resistance genes from non-cultivated lines into elite cultivated lines while avoiding problems with genetically linked deleterious loci or low heritability is a long and often arduous process. In deploying loci derived from wild relatives it is often desirable to introduce a minimal or truncated introgression that provides the desired trait but lacks detrimental effects. To aid introgression reliable marker assays are preferable to phenotypic screens. Success is furthered by simplifying genetics for key attributes to allow focus on genetic gain for quantitative traits such as pest resistance. Moreover, the process of introgressing genomic regions from non-cultivated lines can be greatly facilitated by the availability of accurate markers for MAS.

One of skill in the art would therefore understand that the alleles, polymorphisms, and markers provided by the disclosure allow the tracking and introduction of any of the genomic regions identified herein into any genetic background. In addition, the genomic regions associated with pest resistance disclosed herein can be introgressed from one genotype to another and tracked using MAS. Thus, the inventors' discovery of accurate markers associated with pest resistance will facilitate the development of lettuce plants having beneficial phenotypes. For example, seed can be genotyped using the markers of the present disclosure to select for plants comprising desired genomic regions associated with pest resistance. Moreover, MAS allows identification of plants homozygous or heterozygous for a desired introgression.

Inter-species crosses can also result in suppressed recombination and plants with low fertility or fecundity. For example, suppressed recombination has been observed for the tomato nematode resistance gene Mi, the Mla and Mlg genes in barley, the Yr17 and Lr20 genes in wheat, the Run1 gene in grapevine, and the Rma gene in peanut. Meiotic recombination is essential for classical breeding because it enables the transfer of favorable loci across genetic backgrounds, the removal of deleterious genomic fragments, and pyramiding traits that are genetically tightly linked. Therefore, suppressed recombination forces breeders to enlarge segregating populations for progeny screens in order to arrive at the desired genetic combination.

Phenotypic evaluation of large populations is time-consuming, resource-intensive, and not reproducible in every environment. Marker-assisted selection offers a feasible alternative. Molecular assays designed to detect unique polymorphisms, such as SNPs, are versatile. However, they may fail to discriminate loci within and among lettuce species in a single assay. Structural rearrangements of chromosomes such as deletions impair hybridization and extension of synthetically labeled oligonucleotides. In the case of duplication events, multiple copies are amplified in a single reaction without distinction. The development and validation of accurate and highly predictive markers are therefore essential for successful MAS breeding programs.

### IV. Marker Assisted Breeding and Genetic Engineering Techniques

Genetic markers that can be used in the practice of the present disclosure include, but are not limited to, restriction fragment length polymorphisms (RFLPs), amplified fragment length polymorphisms (AFLPs), simple sequence repeats (SSRs), simple sequence length polymorphisms (SSLPs), single nucleotide polymorphisms (SNPs), insertion/deletion polymorphisms (Indels), variable number tandem repeats (VNTRs), and random amplified polymorphic DNA (RAPD), isozymes, and other markers known to those skilled in the art. Marker discovery and development in crop plants provides the initial framework for applications to marker-assisted breeding activities (U.S. Patent Pub. Nos.: 2005/0204780, 2005/0216545, 2005/0218305, and 2006/00504538). The resulting “genetic map” is the representation of the relative position of characterized loci (polymorphic nucleic acid markers or any other locus for which loci can be identified) to each other.

Polymorphisms comprising as little as a single nucleotide change can be assayed in a number of ways. For example, detection can be made by electrophoretic techniques including a single strand conformational polymorphism (Orita et al. (1989) Genomics, 8 (2), 271-278), denaturing gradient gel electrophoresis (Myers (1985) EP 0273085), or cleavage fragment length polymorphisms (Life Technologies, Inc., Gaithersburg, MD), but the widespread availability of DNA sequencing often makes it easier to simply sequence amplified products directly. Once the polymorphic sequence difference is known, rapid assays can be designed for progeny testing, typically involving some version of PCR amplification of specific loci (PASA; Sommer et al. (1992) Biotechniques 12 (1), 82-87), or PCR amplification of multiple specific loci (PAMSA; Dutton and Sommer (1991) Biotechniques, 11 (6), 700-7002).

Polymorphic markers serve as useful tools for assaying plants for determining the degree of identity of lines or varieties (U.S. Pat. No. 6,207,367). These markers form the basis for determining associations with phenotypes and can be used to drive genetic gain. In certain embodiments of methods of the disclosure, polymorphic nucleic acids can be used to detect in a lettuce plant a genotype associated with pest resistance, identify a lettuce plant with a genotype associated with pest resistance, and to select a lettuce plant with a genotype associated with pest resistance. In certain embodiments of methods of the disclosure, polymorphic nucleic acids can be used to produce a lettuce plant that comprises in its genome an introgressed locus associated with pest resistance. In certain embodiments of the disclosure, polymorphic nucleic acids can be used to breed progeny lettuce plants comprising a locus or loci associated with pest resistance.

Genetic markers may include “dominant” or “codominant” markers. “Codominant” markers reveal the presence of two or more loci (two per diploid individual). “Dominant” markers reveal the presence of only a single locus. Markers are preferably inherited in codominant fashion so that the presence of both loci at a diploid locus, or multiple lociin triploid or tetraploid loci, are readily detectable, and they are free of environmental variation, i.e., their heritability is 1. A marker genotype typically comprises two marker loci at each locus in a diploid organism. The marker allelic composition of each locus can be either homozygous or heterozygous. Homozygosity is a condition where both loci at a locus are characterized by the same nucleotide sequence. Heterozygosity refers to a condition where the two loci at a locus are different.

Nucleic acid-based analyses for determining the presence or absence of the genetic polymorphism (i.e. for genotyping) can be used in breeding programs for identification, selection, introgression, and the like. A wide variety of genetic markers for the analysis of genetic polymorphisms are available and known to those of skill in the art. The analysis may be used to select for genes, portions of genes, QTL, loci, or genomic regions that comprise or are linked to a genetic marker that is linked to or associated with pest resistance in lettuce plants.

As used herein, nucleic acid analysis methods include, but are not limited to, PCR-based detection methods (for example, TaqMan assays), microarray methods, mass spectrometry-based methods and/or nucleic acid sequencing methods, including whole genome sequencing. In certain embodiments, the detection of polymorphic sites in a sample of DNA, RNA, or cDNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis, fluorescence detection methods, or other means.

One method of achieving such amplification employs the polymerase chain reaction (PCR) (Mullis et al. (1986) Cold Spring Harbor Symp. Quant. Biol. 51:263-273; European Patent 50,424; European Patent 84,796; European Patent 258,017; European Patent 237,362; European Patent 201,184; U.S. Pat. Nos. 4,683,202; 4,582,788; and 4,683,194), using primer pairs that are capable of hybridizing to the proximal sequences that define a polymorphism in its double-stranded form. Methods for typing DNA based on mass spectrometry can also be used. Such methods are disclosed in U.S. Pat. Nos. 6,613,509 and 6,503,710, and references found therein.

Polymorphisms in DNA sequences can be detected or typed by a variety of effective methods well known in the art including, but not limited to, those disclosed in U.S. Pat. Nos. 5,468,613, 5,217,863; 5,210,015; 5,876,930; 6,030,787; 6,004,744; 6,013,431; 5,595,890; 5,762,876; 5,945,283; 5,468,613; 6,090,558; 5,800,944; 5,616,464; 7,312,039; 7,238,476; 7,297,485; 7,282,355; 7,270,981 and 7,250,252, all of which are incorporated herein by reference in their entirety. However, the compositions and methods of the present disclosure can be used in conjunction with any polymorphism typing method to detect polymorphisms in genomic DNA samples. These genomic DNA samples used include but are not limited to, genomic DNA isolated directly from a plant, cloned genomic DNA, or amplified genomic DNA.

For instance, polymorphisms in DNA sequences can be detected by hybridization to locus-specific oligonucleotide (ASO) probes as disclosed in U.S. Pat. Nos. 5,468,613 and 5,217,863. U.S. Pat. No. 5,468,613 discloses locus specific oligonucleotide hybridizations where single or multiple nucleotide variations in nucleic acid sequence can be detected in nucleic acids by a process in which the sequence containing the nucleotide variation is amplified, spotted on a membrane, and treated with a labeled sequence-specific oligonucleotide probe.

Target nucleic acid sequence can also be detected by probe ligation methods, for example as disclosed in U.S. Pat. No. 5,800,944 where sequence of interest is amplified and hybridized to probes followed by ligation to detect a labeled part of the probe.

Microarrays can also be used for polymorphism detection, wherein oligonucleotide probe sets are assembled in an overlapping fashion to represent a single sequence such that a difference in the target sequence at one point would result in partial probe hybridization (Borevitz et al., Genome Res. 13:513-523 (2003); Cui et al., Bioinformatics 21:3852-3858 (2005). On any one microarray, it is expected there will be a plurality of target sequences, which may represent genes and/or noncoding regions wherein each target sequence is represented by a series of overlapping oligonucleotides, rather than by a single probe. This platform provides for high throughput screening of a plurality of polymorphisms. Typing of target sequences by microarray-based methods is described in U.S. Pat. Nos. 6,799,122; 6,913,879; and 6,996,476.

Other methods for detecting SNPs and Indels include single base extension (SBE) methods. Examples of SBE methods include, but are not limited, to those disclosed in U.S. Pat. Nos. 6,004,744; 6,013,431; 5,595,890; 5,762,876; and 5,945,283.

In another method for detecting polymorphisms, SNPs and Indels can be detected by methods disclosed in U.S. Pat. Nos. 5,210,015; 5,876,930; and 6,030,787 in which an oligonucleotide probe having a 5′ fluorescent reporter dye and a 3′ quencher dye covalently linked to the 5′ and 3′ ends of the probe. When the probe is intact, the proximity of the reporter dye to the quencher dye results in the suppression of the reporter dye fluorescence, e.g. by Forster-type energy transfer. During PCR, forward and reverse primers hybridize to a specific sequence of the target DNA flanking a polymorphism while the hybridization probe hybridizes to polymorphism-containing sequence within the amplified PCR product. In the subsequent PCR cycle, DNA polymerase with 5′→3′ exonuclease activity cleaves the probe and separates the reporter dye from the quencher dye resulting in increased fluorescence of the reporter.

In another embodiment, a locus or loci of interest can be directly sequenced using nucleic acid sequencing technologies. Methods for nucleic acid sequencing are known in the art and include technologies provided by 454 Life Sciences (Branford, CT), Agencourt Bioscience (Beverly, MA), Applied Biosystems (Foster City, CA), LI-COR Biosciences (Lincoln, NE), NimbleGen Systems (Madison, WI), Illumina (San Diego, CA), and VisiGen Biotechnologies (Houston, TX). Such nucleic acid sequencing technologies comprise formats such as parallel bead arrays, sequencing by ligation, capillary electrophoresis, electronic microchips, “biochips,” microarrays, parallel microchips, and single-molecule arrays.

Various genetic engineering technologies have been developed and may be used by those of skill in the art to introduce traits in plants. In certain aspects of the disclosure, traits are introduced into lettuce plants via altering or introducing a single genetic locus or transgene into the genome of a variety or progenitor thereof. Methods of genetic engineering to modify, delete, or insert genes and polynucleotides into the genomic DNA of plants are well-known in the art.

In specific embodiments of the disclosure, improved lettuce lines can be created through the site-specific modification of a plant genome. Methods of genetic engineering include, for example, utilizing sequence-specific nucleases such as zinc-finger nucleases (see, for example, U.S. Pat. Appl. Pub. No. 2011/0203012); engineered or native meganucleases; TALE-endonucleases (see, for example, U.S. Pat. Nos. 8,586,363 and 9,181,535); and RNA-guided endonucleases, such as those of the CRISPR/Cas systems (see, for example, U.S. Pat. Nos. 8,697,359 and 8,771,945 and U.S. Pat. Appl. Pub. No. 2014/0068797). One embodiment of the disclosure thus relates to utilizing a nuclease or any associated protein to carry out genome modification. This nuclease could be provided heterologously within donor template DNA for templated-genomic editing or in a separate molecule or vector. A recombinant DNA construct may also comprise a sequence encoding one or more guide RNAs to direct the nuclease to the site within the plant genome to be modified. Further methods for altering or introducing a single genetic locus include, for example, utilizing single-stranded oligonucleotides to introduce base pair modifications in a plant genome (see, for example Sauer et al., Plant Physiol, 170 (4): 1917-1928, 2016).

Methods for site-directed alteration or introduction of a single genetic locus are well-known in the art and include those that utilize sequence-specific nucleases, such as the aforementioned, or complexes of proteins and guide-RNA that cut genomic DNA to produce a double-strand break (DSB) or nick at a genetic locus. As is well-understood in the art, during the process of repairing the DSB or nick introduced by the nuclease enzyme, a donor template, transgene, or expression cassette polynucleotide may become integrated into the genome at the site of the DSB or nick. The presence of homology arms in the DNA to be integrated may promote the adoption and targeting of the insertion sequence into the plant genome during the repair process through homologous recombination or non-homologous end joining (NHEJ).

In another embodiment of the disclosure, genetic transformation may be used to insert a selected transgene into a plant of the disclosure or may, alternatively, be used for the preparation of transgenes which can be introduced by backcrossing. Methods for the transformation of plants that are well-known to those of skill in the art and applicable to many crop species include, but are not limited to, electroporation, microprojectile bombardment, Agrobacterium-mediated transformation, and direct DNA uptake by protoplasts.

To effect transformation by electroporation, one may employ either friable tissues, such as a suspension culture of cells or embryogenic callus or alternatively one may transform immature embryos or other organized tissue directly. In this technique, one would partially degrade the cell walls of the chosen cells by exposing them to pectin-degrading enzymes (pectolyases) or mechanically wound tissues in a controlled manner.

An efficient method for delivering transforming DNA segments to plant cells is microprojectile bombardment. In this method, particles are coated with nucleic acids and delivered into cells by a propelling force. Exemplary particles include those comprised of tungsten, platinum, and preferably, gold. For the bombardment, cells in suspension are concentrated on filters or solid culture medium. Alternatively, immature embryos or other target cells may be arranged on solid culture medium. The cells to be bombarded are positioned at an appropriate distance below the microprojectile stopping plate.

An illustrative embodiment of a method for delivering DNA into plant cells by acceleration is the Biolistics Particle Delivery System, which can be used to propel particles coated with DNA or cells through a screen, such as a stainless steel or Nytex screen, onto a surface covered with target cells. The screen disperses the particles so that they are not delivered to the recipient cells in large aggregates. Microprojectile bombardment techniques are widely applicable and may be used to transform virtually any plant species.

Agrobacterium-mediated transfer is another widely applicable system for introducing gene lociinto plant cells. An advantage of the technique is that DNA can be introduced into whole plant tissues, thereby bypassing the need for regeneration of an intact plant from a protoplast. Modern Agrobacterium transformation vectors are capable of replication in E. coli as well as Agrobacterium, allowing for convenient manipulations (Klee et al., Nat. Biotechnol., 3 (7): 637-642, 1985). Moreover, recent technological advances in vectors for Agrobacterium-mediated gene transfer have improved the arrangement of genes and restriction sites in the vectors to facilitate the construction of vectors capable of expressing various polypeptide coding genes. The vectors described have convenient multi-linker regions flanked by a promoter and a polyadenylation site for direct expression of inserted polypeptide coding genes. Additionally, Agrobacterium containing both armed and disarmed Ti genes can be used for transformation.

In those plants where Agrobacterium-mediated transformation is efficient, it is the method of choice because of the facile and defined nature of the gene locus transfer. The use of Agrobacterium-mediated plant integrating vectors to introduce DNA into plant cells is well known in the art (Fraley et al., Nat. Biotechnol., 3:629-635, 1985; U.S. Pat. No. 5,563,055).

Transformation of plant protoplasts also can be achieved using methods based on calcium phosphate precipitation, polyethylene glycol treatment, electroporation, and combinations of these treatments (see, for example, Potrykus et al., Mol. Gen. Genet., 199:183-188, 1985; Omirulleh et al., Plant Mol. Biol., 21 (3): 415-428, 1993; Fromm et al., Nature, 312:791-793, 1986; Uchimiya et al., Mol. Gen. Genet., 204:204, 1986; Marcotte et al., Nature, 335:454, 1988). Transformation of plants and expression of foreign genetic elements is exemplified in Choi et al. (Plant Cell Rep., 13:344-348, 1994), and Ellul et al. (Theor. Appl. Genet., 107:462-469, 2003).

### V. Definitions

The following definitions are provided to better define the present disclosure and to guide those of ordinary skill in the art in the practice of the present disclosure. Unless otherwise noted, terms are to be understood according to conventional usage by those of ordinary skill in the relevant art.

As used herein, the term “plant” includes plant cells, plant protoplasts, plant cells of tissue culture from which lettuce plants can be regenerated, plant calli, plant clumps and plant cells that are intact in plants or parts of plants such as pollen, flowers, seeds, leaves, stems, or any portion thereof, or a non-regenerable portion of a plant part, including a cell, for example. As used in this context, a “non-regenerable” portion of a plant part is a portion that cannot be induced to form a whole plant or that cannot be induced to form a whole plant that is capable of sexual and/or asexual reproduction. In certain non-limiting embodiments, a non-regenerable portion of a plant or part thereof is a seed, leaf, flower, stem, root or cell, or any portion thereof.

As used herein, the term “population” means a genetically heterogeneous collection of plants that share a common parental derivation.

As used herein, the terms “variety” and “cultivar” mean a group of similar plants that by their genetic pedigrees and performance can be identified from other varieties within the same species.

As used herein, an “allele” refers to one of two or more alternative forms of a genomic sequence at a given locus on a chromosome.

A “quantitative trait locus” (QTL) is a chromosomal location that encodes for at least a first locus that affects the expressivity of a phenotype.

As used herein, a “marker” means a detectable characteristic that can be used to discriminate between organisms. Examples of such characteristics include, but are not limited to, genetic markers, biochemical markers, metabolites, morphological characteristics, and agronomic characteristics.

As used herein, the term “phenotype” means the detectable characteristics of a cell or organism that can be influenced by gene expression.

As used herein, the term “genotype” means the specific allelic makeup of a plant.

As used herein, “elite” or “cultivated” variety means any variety that has resulted from breeding and selection for superior agronomic performance. An “elite plant” refers to a plant belonging to an elite variety. Numerous elite varieties are available and known to those of skill in the art of lettuce breeding. An “elite population” is an assortment of elite individuals or varieties that can be used to represent the state of the art in terms of agronomically superior genotypes of a given crop species, such as lettuce. Similarly, an “elite germplasm” or elite strain of germplasm is an agronomically superior germplasm.

As used herein, the term “isolated” refers to at least partially separating a molecule (e.g. an isolated nucleic acid molecule or isolated recombinant DNA segment) from other molecules typically associated with it in its natural state.

As used herein, the term “introgressed,” when used in reference to a genetic locus, refers to a genetic locus that has been introduced into a new genetic background, such as through backcrossing. Introgression of a genetic locus can be achieved through plant breeding methods and/or by molecular genetic methods. Such molecular genetic methods include, but are not limited to, various plant transformation techniques and/or methods that provide for homologous recombination, non-homologous recombination, site-specific recombination, and/or genomic modifications that provide for locus substitution or locus conversion.

As used herein, the terms “recombinant” or “recombined” in the context of a chromosomal segment refer to recombinant DNA sequences comprising one or more genetic lociin a configuration in which they are not found in nature, for example as a result of a recombination event between homologous chromosomes during meiosis.

As used herein, the term “linked,” when used in the context of nucleic acid markers and/or genomic regions, means that the markers and/or genomic regions are located on the same linkage group or chromosome such that they tend to segregate together at meiosis.

As used herein, “tolerance locus” means a locus associated with tolerance or resistance to disease or pest. For instance, a tolerance locus according to the present disclosure may, in one embodiment, control tolerance or susceptibility to downy mildew disease.

As used herein, “tolerance” or “improved tolerance” in a plant refers to the ability of the plant to perform well, for example by maintaining yield, under disease conditions or upon pest infestations. Tolerance may also refer to the ability of a plant to maintain a plant vigor phenotype under disease conditions or under pest infestations. Tolerance is a relative term, indicating that a “tolerant” plant is more able to maintain performance compared to a different (less tolerant) plant (e. g. a different plant variety) grown in similar disease conditions or under similar pest pressure. One of skill will appreciate that plant tolerance to disease or pest conditions varies widely and can represent a spectrum of more-tolerant or less-tolerant phenotypes. However, by simple observation, one of skill can generally determine the relative tolerance of different plants, plant varieties, or plant families under disease or pest conditions, and furthermore, will also recognize the phenotypic gradations of “tolerance.”

As used herein “resistance” or “improved resistance” in a plant to disease or pest conditions is an indication that the plant is more able to reduce disease or pest burden than a non-resistant or less resistant plant. Resistance is a relative term, indicating that a “resistant” plant is more able to reduce disease burden or pest burden compared to a different (less resistant) plant (e. g., a different plant variety) grown in similar disease conditions or pest pressure. One of skill will appreciate that plant resistance to disease conditions or pest infestation varies widely and can represent a spectrum of more-resistant or less-resistant phenotypes. However, by simple observation, one of skill can generally determine the relative resistance of different plants, plant varieties, or plant families under disease conditions or pest pressure, and furthermore, will also recognize the phenotypic gradations of “resistant.”

As used herein, “resistance allele” means the nucleic acid sequence associated with tolerance or resistance to pest infestation.

“Sequence identity” and “sequence similarity” can be determined by alignment of two nucleotide sequences using global or local alignment algorithms. Sequences may then be referred to as “substantially identical” or “essentially similar” when they are optimally aligned by for example the programs GAP or BESTFIT or the Emboss program “Needle” (using default parameters) share at least a certain minimal percentage of sequence identity. These programs use the Needleman and Wunsch global alignment algorithm to align two sequences over their entire length, maximizing the number of matches and minimizing the number of gaps. Generally, the default parameters are used, with a gap creation penalty=10 and gap extension penalty=0.5 (both for nucleotide and protein alignments). For nucleotides the default scoring matrix used is DNAFULL (Henikoff & Henikoff, PNAS 89:10915-10919; 1992). Sequence alignments and scores for percentage sequence identity may for example be determined using computer programs, such as EMBOSS as available on the world wide web under ebi.ac.uk/Tools/psa/emboss_needle. Alternatively, sequence similarity or identity may be determined by searching against databases such as FASTA, BLAST, etc., but hits should be retrieved and aligned pairwise to compare sequence identity. Two nucleic acid sequences have “substantial sequence identity” if the percentage sequence identity is at least 85%, 90%, 95%, 98%, 99% or more (e.g. at least 99.1, 99.2, 99.3, 99.4, 99.5, 99.6, 99.7, 99.8, 99.9 (as determined by Emboss “needle” using default parameters, i.e. gap creation penalty=10, gap extension penalty=0.5, using scoring matrix DNAFULL for nucleic acids)). Markers may sometimes exhibit variation, particularly in regions which are not recognized by the probes.

The term “about” is used to indicate that a value includes the standard deviation of error for the device or method being employed to determine the value. The use of the term “or” in the claims is used to mean “and/or” unless explicitly indicated to refer to alternatives only or the alternatives are mutually exclusive, although the disclosure supports a definition that refers to only alternatives and to “and/or.” When used in conjunction with the word “comprising” or other open language in the claims, the words “a” and “an” denote “one or more,” unless specifically noted. The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps. Similarly, any plant that “comprises,” “has” or “includes” one or more traits is not limited to possessing only those one or more traits and covers other unlisted traits.

### VI. Deposit Information

A deposit was made of at least 625 seeds of each of lettuce line SVLD0093, lettuce line LAC23B, and lettuce line CHD-8W22-6051V. The deposits were made with the Provasoli-Guillard National Center for Marine Algae and Microbiota (NCMA), 60 Bigelow Drive, East Boothbay, Maine, 04544, USA. The deposits are assigned NCMA Accession Nos. 202212295, 202307003, and 202306045, respectively, and the dates of deposit were Dec. 22, 2022, Jul. 28, 2023, and Jun. 14, 2023. Access to the deposits will be available during the pendency of the application to persons entitled thereto upon request. The deposits have been accepted under the Budapest Treaty and will be maintained in the NCMA depositary, which is a public depository, for a period of 30 years, or 5 years after the most recent request, or for the enforceable life of the patent, whichever is longer, and will be replaced if nonviable during that period. Applicant does not waive any infringement of their rights granted under this patent or any other form of variety protection, including the Plant Variety Protection Act (7 U.S.C. 2321 et seq.).

### Embodiments

For further illustration, additional exemplary, non-limiting embodiments of the present disclosure are set forth below.

Embodiment 1 relates to a recombinant DNA segment comprising a Bremia lactucae resistance allele from Lactuca serriola, wherein said DNA segment comprises the sequence of marker locus M1 (SEQ ID NO: 1) and marker locus M2 (SEQ ID NO: 2).

Embodiment 2 relates to the recombinant DNA segment of embodiment 1, wherein: a) said recombinant DNA segment further comprises a marker locus sequence selected from the group consisting of marker locus M3 (SEQ ID NO: 3), marker locus M4 (SEQ ID NO: 4), marker locus M5 (SEQ ID NO: 5), and marker locus M6 (SEQ ID NO: 6); b) the recombinant DNA segment is further defined as comprised within a plant, plant part, plant cell, or seed; or c) a representative sample of seed comprising said recombinant DNA segment has been deposited under NCMA Accession No. 202212295, NCMA Accession No. 202307003, or NCMA Accession No. 202306045.

Embodiment 3 relates to the recombinant DNA segment of any one of embodiments 1 to 2, wherein said recombinant DNA segment further comprises: a) marker locus M3 (SEQ ID NO: 3), M4 (SEQ ID NO: 4), and M6 (SEQ ID NO: 6); b) marker locus M5 (SEQ ID NO: 5), and M6 (SEQ ID NO: 6); c) marker locus M3 (SEQ ID NO: 3), and M6 (SEQ ID NO: 6); or d) marker locus M5 (SEQ ID NO: 5).

Embodiment 4 relates to a method of identifying a Lactuca sativa plant exhibiting resistance to Bremia lactucae, wherein: a) a population of progeny plants having a parent comprising resistance to Bremia lactucae is obtained; and wherein the method further comprises b) screening said population with at least one nucleic acid marker to detect a polymorphism genetically linked to Bremia lactucae resistance; and c) identifying in said population at least a first progeny plant comprising a haplotype associated with Bremia lactucae resistance, wherein the haplotype comprises a Bremia lactucae resistance allele from Lactuca serriola at a locus flanked in the genome of said plant by marker locus M1 (SEQ ID NO:1) and marker locus M2 (SEQ ID NO:2) on chromosome 9.

Embodiment 5 relates to the method of identifying a Lactuca sativa plant exhibiting resistance to Bremia lactucae of embodiment 4, wherein identifying said progeny plant comprises: a) detecting a marker locus within or genetically linked to a chromosomal segment flanked in the genome of said plant by marker locus M1 (SEQ ID NO: 1) and marker locus M2 (SEQ ID NO: 2) on chromosome 9; b) detecting at least one polymorphism at a locus selected from the group consisting of M1 (SEQ ID NO: 1), marker locus M2 (SEQ ID NO: 2), marker locus M3 (SEQ ID NO: 3), marker locus M4 (SEQ ID NO: 4), marker locus M5 (SEQ ID NO: 5), and marker locus M6 (SEQ ID NO: 6); or d) detecting at least one polymorphism at marker locus M5 (SEQ ID NO: 5).

Embodiment 6 relates to the method of identifying a Lactuca sativa plant exhibiting resistance to Bremia lactucae of embodiments 4 or 5, wherein the haplotype comprises a Bremia lactucae resistance allele from Lactuca serriola at a locus flanked in the genome of said plant by marker locus M1 (SEQ ID NO:1) and marker locus M6 (SEQ ID NO:6) on chromosome 9.

Embodiment 7 relates to the method of identifying a Lactuca sativa plant exhibiting resistance to Bremia lactucae of any one of embodiments 4 to 6, wherein screening said population comprises PCR, single strand conformational polymorphism analysis, denaturing gradient gel electrophoresis, cleavage fragment length polymorphism analysis, TAQMAN assay, and/or DNA sequencing.

Embodiment 8 relates to the method of identifying a Lactuca sativa plant exhibiting resistance to Bremia lactucae of any one of embodiments 4 to 7, wherein a representative sample of seed comprising said Bremia lactucae resistance allele has been deposited under NCMA Accession No. 202212295, NCMA Accession No. 202307003, or NCMA Accession No. 202306045.

Embodiment 9 relates to a method for identifying a lettuce plant comprising a Bremia lactucae resistance allele from Lactuca serriola, comprising: a) obtaining nucleic acids from at least a first lettuce plant; and b) identifying in said nucleic acids the presence of at least a first genetic marker indicative of the presence of a chromosomal segment flanked in the genome of said plant by marker locus M1 (SEQ ID NO:1) and marker locus M2 (SEQ ID NO:2) on chromosome 9; wherein said chromosomal segment comprises a Bremia lactucae resistance allele conferring to said plant increased resistance to Bremia lactucae compared to a plant not comprising said allele.

Embodiment 10 relates to the method for identifying a lettuce plant comprising a Bremia lactucae resistance allele from Lactuca serriola of embodiment 9, wherein said identifying comprises detecting a marker genetically linked to M1 (SEQ ID NO: 1), marker locus M2 (SEQ ID NO: 2), marker locus M3 (SEQ ID NO: 3), marker locus M4 (SEQ ID NO: 4), marker locus M5 (SEQ ID NO: 5), or marker locus M6 (SEQ ID NO: 6).

Embodiment 11 relates to the method for identifying a lettuce plant comprising a Bremia lactucae resistance allele from Lactuca serriola of embodiment 9 or 10, wherein the first genetic marker is indicative of the presence of a chromosomal segment flanked in the genome of said plant by marker locus M1 (SEQ ID NO:1) and marker locus M6 (SEQ ID NO:6) on chromosome 9.

Embodiment 12 relates to a method of selecting a lettuce plant or lettuce seed, said method comprising: a) detecting in a population of lettuce plants or lettuce seeds a lettuce plant or lettuce seed comprising an allele from Lactuca serriola that confers to said plant resistance to Bremia lactucae compared to a plant not comprising said allele; and b) selecting said lettuce plant or lettuce seed comprising said allele from Lactuca serriola that confers to said plant a resistance to Bremia lactucae.

Embodiment 13 relates to the method of selecting a lettuce plant or lettuce seed of embodiment 12, wherein said allele is located on chromosome 9.

Embodiment 14 relates to the method of selecting a lettuce plant or lettuce seed of embodiment 12 or 13, wherein said allele is linked to any marker selected from the group consisting of marker M1 (SEQ ID NO: 1), marker locus M2 (SEQ ID NO: 2), marker locus M3 (SEQ ID NO: 3), marker locus M4 (SEQ ID NO: 4), marker locus M5 (SEQ ID NO: 5), or marker locus M6 (SEQ ID NO: 6) on chromosome 9.

Embodiment 15 relates to the method of selecting a lettuce plant or lettuce seed of any one of embodiments 12 to 14, wherein said plant is homozygous for said allele from Lactuca serriola.

Embodiment 16 relates to the method of selecting a lettuce plant or lettuce seed of any one of embodiments 12 to 15, wherein said plant is heterozygous for said allele from Lactuca serriola.

Embodiment 17 relates to the method of selecting a lettuce plant or lettuce seed of any one of embodiments 12 to 16, wherein said allele is linked to: a) marker locus M3 (SEQ ID NO: 3), M4 (SEQ ID NO: 4), and M6 (SEQ ID NO: 6); b) marker locus M5 (SEQ ID NO: 5) and M6 (SEQ ID NO: 6); c) marker locus M3 (SEQ ID NO: 3) and M6 (SEQ ID NO: 6); or d) marker locus M5 (SEQ ID NO: 5).

Embodiment 18 relates to the method of selecting a lettuce plant or lettuce seed of any one of embodiments 12 to 17, wherein: marker locus M3 comprises a C at position 106 of SEQ ID NO:3; marker locus M4 comprises an A at position 61 of SEQ ID NO:4; marker locus M5 comprises an A at position 301 of SEQ ID NO:5; or marker locus M6 comprises a C at position 101 of SEQ ID NO:6.

Embodiment 19 relates to the method of selecting a lettuce plant or lettuce seed of any one of embodiments 12 to 18, wherein said lettuce plant or lettuce seed is an elite plant or a seed of an elite plant.

Embodiment 20 relates to a nucleic acid molecule comprising an allele conferring to a lettuce plant resistance to Bremia lactucae compared to a plant not comprising said allele, wherein the nucleic acid molecule is obtainable or can be obtained from seed deposited under NCMA Accession No. 202212295, NCMA Accession No. 202307003, or NCMA Accession No. 202306045, and wherein the nucleic acid molecule comprises at least one marker selected from the group consisting of marker locus M3 (SEQ ID NO: 3), marker locus M4 (SEQ ID NO: 4), marker locus M5 (SEQ ID NO: 5), and marker locus M6 (SEQ ID NO: 6).

Embodiment 21 relates to the nucleic acid molecule comprising an allele conferring to a lettuce plant resistance to Bremia lactucae compared to a plant not comprising said allele of embodiment 20, wherein said nucleic acid molecule comprises: a) marker locus M3 (SEQ ID NO: 3), M4 (SEQ ID NO: 4), and M6 (SEQ ID NO: 6); b) marker locus M5 (SEQ ID NO: 5) and M6 (SEQ ID NO: 6); c) marker locus M3 (SEQ ID NO: 3) and M6 (SEQ ID NO: 6); or d) marker locus M5 (SEQ ID NO: 5).

Embodiment 22 relates to the nucleic acid molecule comprising an allele conferring to a lettuce plant resistance to Bremia lactucae compared to a plant not comprising said allele of embodiment 20 or 21, wherein the at least one marker locus comprises: SEQ ID NO:3 comprising a C at position 106; SEQ ID NO:4 comprising an A at position 61; SEQ ID NO:5 comprising an A at position 301; or SEQ ID NO:6 comprising a C at position 101.

Embodiment 23 relates to the method of selecting a lettuce plant or lettuce seed of any one of embodiments 20 to 22, wherein the nucleic acid molecule further comprises at least one marker selected from the group consisting of marker M1 (SEQ ID NO: 1) and marker locus M2 (SEQ ID NO: 2).

Embodiment 24 relates to the method of selecting a lettuce plant or lettuce seed of any one of embodiments 20 to 22, wherein the at least one marker locus comprises: SEQ ID NO:1 comprising an A at position 61; SEQ ID NO:1 comprising a G at position 61; SEQ ID NO:2 comprising an A at position 61; or SEQ ID NO:2 comprising a G at position 61.

### EXAMPLES

The embodiments described herein may be more readily understood through reference to the following examples, which are provided by way of illustration, and are not intended to be limiting, unless specified. It should be appreciated by those of skill in the art that the techniques disclosed in the following examples represent techniques discovered by the inventors to function well in the practice of this disclosure. However, those of skill in the art should, in light of the disclosure, appreciate that many changes can be made in the specific embodiments that are disclosed and still obtain a like or similar result without departing from the principles of the disclosure, therefore all matter set forth or shown in the accompanying drawings is to be interpreted as illustrative and not in a limiting sense.

### Example 1. Testing for Downy Mildew Resistance in Lettuce

Pathology tests for determining resistance to downy mildew can be carried out, for example, using differential sets of lettuce varieties provided by the International Bremia Evaluation Board (IBEB) that define resistance to different B. lactucae races. A lettuce variety that is commonly used as a susceptible control is the variety ‘Green Towers,’ however any other susceptible varieties may be used as controls in a pathology test. The resistant control used in the test depends on the B. lactucae isolate that is being used, but the differential set generally offers multiple resistant variety options per downy mildew isolate. See Table 1. The IBEB initiative provides contacts where validated seeds of the varieties of the differential set can be obtained as well as validated samples of B. lactucae isolates.

TABLE 1
Differential sets to characterize isolates of Bremia lactucae as defined by IBEB

| | | | | | | | | | | RYZ | |
|---|---|---|---|---|---|---|---|---|---|---|---|
| | | | | UC | | | | | | 2164 | |
| | Green- | Dandie | R4T57D | Dm14 | NunDm15 | CGDm16 | Colorado | FrRsal-1 | Argeles | Dm24/ | RYZ910457 |
| | Towers | Dm3 | Dm4 | Dm14 | Dm15 | Dm16 | Dm18 | Rsal-1 | R38 | 38 | R52 |
| | ID | | | | | | | | | | |
| | 0 | S1 | S2 | S3 | S4 | S5 | S6 | S7 | S8 | S9 | S10 |
| | Sextet value | | | | | | | | | | |
| | | 1 | 2 | 4 | 8 | 16 | 32 | 1 | 2 | 4 | 8 |
| Bl: 7US | + | + | − | + | + | + | + | − | − | − | − |
| Bl: 8US | + | + | + | + | + | + | + | − | − | − | − |
| Bl: 9US | + | + | − | + | + | + | + | + | − | − | + |
| Bl: 16EU | + | + | + | − | − | + | − | − | − | − | − |
| Bl: 17EU | + | + | − | + | + | − | + | + | − | − | − |
| Bl: 18EU | + | − | + | − | − | + | + | − | − | − | − |
| Bl: 20EU | + | + | + | − | − | + | + | − | − | − | − |
| BI: 21EU | + | + | + | − | + | + | − | + | − | − | − |
| Bl: 22EU | + | − | + | + | + | − | + | − | − | − | − |
| Bl: 23EU | + | + | + | − | − | + | − | − | + | − | − |
| Bl: 24EU | + | − | + | − | − | + | + | − | + | − | − |
| Bl: 25EU | + | − | + | − | − | + | + | + | − | − | − |
| Bl: 26EU | + | + | + | − | − | + | + | + | + | − | − |
| Bl: 27EU | + | + | + | + | + | − | + | − | + | + | − |
| Bl: 29EU | + | − | + | + | + | + | + | + | + | + | − |
| Bl: 30EU | + | − | + | + | + | − | + | − | + | + | − |
| Bl: 31EU | + | + | + | + | − | − | + | − | − | + | + |
| Bl: 32EU | + | + | + | − | + | + | − | − | − | − | − |
| Bl: 33EU | + | − | + | + | + | + | + | + | + | + | − |
| Bl: 34EU | + | − | + | + | − | + | + | + | + | + | + |
| Bl: 35EU | + | − | + | + | + | + | + | + | + | + | + |
| Bl: 36EU | + | + | + | + | − | + | + | + | + | + | + |
| Bl: 37EU | + | − | + | + | + | − | + | + | + | + | + |
| Bl: 38EU | + | − | + | + | + | − | + | + | + | + | + |
| Bl: 39EU | + | + | + | + | − | + | + | + | + | + | + |
| Bl: 40EU | + | - | + | + | + | + | + | + | + | + | + |

| | | | | | Kibrille | | | RYZ20007 | | |
|---|---|---|---|---|---|---|---|---|---|---|
| | Bedford | Balesta | Bartoli | Design | Dm11, | Fenston | Bataille | Dm11, | | |
| | R53 | R54 | R55 | R56 | R57 | R65 | R59 | R58 | | |
| | ID | | | | | | | | | |
| | S11 | S12 | S13 | S14 | S15 | S16 | S17 | S18 | | D |
| | Sextet value | | | | | | | | | sextet |
| | 16 | 32 | 1 | 2 | 4 | 8 | 16 | 32 | Set | code |
| Bl: 7US | | − | − | | − | − | − | − | D | 61-.-. |
| Bl: 8US | | − | − | + | − | − | + | − | D | 63-.-18 |
| Bl: 9US | (−) | − | − | + | − | − | − | − | D | 61-09-02 |
| Bl: 16EU | − | − | − | − | − | − | − | − | D | 19-00-00 |
| Bl: 17EU | (+) | − | − | − | − | − | − | − | D | 45-17-00 |
| Bl: 18EU | − | − | − | − | − | − | − | − | D | 50-00-00 |
| Bl: 20EU | − | − | − | − | − | − | − | − | D | 51-00-00 |
| BI: 21EU | − | − | − | − | − | − | − | − | D | 27-01-00 |
| Bl: 22EU | − | + | − | − | − | − | − | − | D | 46-32-00 |
| Bl: 23EU | − | − | − | − | − | − | − | − | D | 19-02-00 |
| Bl: 24EU | − | − | − | (−) | − | (−) | (−) | − | D | 50-02-00 |
| Bl: 25EU | − | − | − | − | − | − | − | − | D | 50-01-00 |
| Bl: 26EU | − | − | − | − | − | − | − | − | D | 51-03-00 |
| Bl: 27EU | − | + | − | − | − | − | − | − | D | 47-38-00 |
| Bl: 29EU | − | − | − | − | − | − | − | − | D | 62-07-00 |
| Bl: 30EU | − | − | − | + | − | − | − | − | D | 46-06-02 |
| Bl: 31EU | − | − | − | + | − | − | − | − | D | 39-12-02 |
| Bl: 32EU | − | − | − | − | + | − | − | − | D | 27-00-04 |
| Bl: 33EU | − | − | − | + | + | − | − | − | D | 62-07-06 |
| Bl: 34EU | − | − | + | (−) | − | − | − | − | D | 54-15-01 |
| Bl: 35EU | − | − | − | + | + | − | − | − | D | 62-15-06 |
| Bl: 36EU | − | − | + | − | − | − | − | − | D | 55-15-01 |
| Bl: 37EU | − | − | − | + | + | + | − | − | D | 46-15-14 |
| Bl: 38EU | − | − | − | + | + | − | − | + | D | 46-15-38 |
| Bl: 39EU | − | − | + | − | − | − | − | + | D | 55-15-33 |
| Bl: 40EU | + | − | + | (−) | − | − | − | − | D | 62-31-01 |

A pathology test ideally evaluates between 15-30 plants per variety. The experiment can be performed with plants grown in soil or on artificial substrate containing plant growth medium, e.g. 0.5× Hoagland solution. The plants are ideally germinated and grown at a temperature of 15° C. with 12 hours of light. After about 7 days, when the cotyledons have fully opened, the plants are inoculated with B. lactucae conidia by spraying them with inoculum suspension. The inoculum suspension is produced by washing leaves containing a sporulating B. lactucae infection, which is about 7-10 days after inoculation, with sterile water, sieved to remove remaining plant parts, and diluted with sterile water to a final concentration of 1×10⁴ conidia/ml.

After inoculation, the plants are maintained in an environment with very high humidity (around 100%) at about 15° C. with 12-16 hours of light. Seven to 10 days post inoculation, the first sporulation should be visible. The first reading in the experiment should be done nine to 10 days post-inoculation. A second reading should be performed 12 to 14 days post-inoculation. A 1-9 rating scale can be used to score the level of resistance observed in the experiment, where a score of 1 indicates no infection and a score of 9 indicates heavy sporulation. Intermediate scores are as follows: 2 indicates observable necrosis only; 3 indicates observable necrosis and mild sporulation; and 7 indicates moderate sporulation. A rating of 1-3 indicates resistance to B. lactucae; and a rating of 7-9 indicates susceptibility to B. lactucae. A similar scoring scale has been developed by and is available from the IBEB. The experiment is considered successful when the susceptible control is sporulating and the other controls score as expected.

### Example 2. Identification of Novel Alleles Conferring Resistance to DM

200 Lactuca serriola accessions from a public genebank were screened for their resistance to Bremia lactucae isolates Bl: 1-24EU. It was hypothesized that lines resistant to six or more Bremia isolates would be more likely to have resistance to future isolates. Based on these metrics, BC1 creation was initiated; and F1 crosses were made with recurrent parent representatives of the Butterhead and Crisphead lettuce types.

Phenotyping of each BC1F3 was carried out in accordance with Example 1. Interestingly, comparison of the resistance pattern observed in certain BC1F3 lines against the known phenotypes of the recurrent parents indicated that novel alleles on chromosome 9 were identified. BC1F3 lines were selected for the novel resistance allele and selfed to create introgression lines. Using the L. sativa v8 public genome (Reyes et al 2017) as a reference, informative markers were determined based on the combination of donor and the recurrent parent employed for breeding.

Lactuca sativa line SVLD0093 comprises a novel B. lactucae resistance allele from L. serriola on chromosome 9 flanked in the genome of said plant by marker locus M1 (SEQ ID NO: 1) and marker locus M2 (SEQ ID NO: 2), which may also be tracked using one or more of marker locus marker locus M3 (SEQ ID NO: 3), marker locus M6 (SEQ ID NO: 6), and marker locus M7 (SEQ ID NO: 7).

Lactuca serriola line LAC23B comprises a novel B. lactucae resistance allele on chromosome 9 flanked in the genome of said plant by marker locus M1 (SEQ ID NO: 1) and marker locus M2 (SEQ ID NO: 2), which may also be tracked using one or more of marker locus M5 (SEQ ID NO: 5), marker locus M6 (SEQ ID NO: 6), and marker locus M7 (SEQ ID NO: 7).

Lactuca sativa line CHD-8W22-6051V comprises a novel B. lactucae resistance allele from L. serriola on chromosome 9 flanked in the genome of said plant by marker locus M1 (SEQ ID NO: 1) and marker locus M2 (SEQ ID NO: 2), which may also be tracked using one or more of marker locus M3 (SEQ ID NO: 3), marker locus M4 (SEQ ID NO: 4), marker locus M6 (SEQ ID NO: 6), and marker locus M7 (SEQ ID NO: 7). The three alleles from different L. serriola sources described herein map to the same resistance locus and all three alleles are located between marker locus M1 and marker locus M2. In addition, one or more of the following marker loci: M3, M4, M5, M6 and M7 can be used to track that the relevant allele on chromosome 9 has been successfully introgressed in a cultivated lettuce plant. The markers that can be used to track the resistance allele(s) from Lactuca serriola described herein are shown in Table 2 below.

The marker information provided herein also allows for identification of additional L. serriola accessions that could potentially be used as a donor source for DM resistance; reducing the time and costs associated with screening large number of genebank accessions. L. serriola germplasm is available from public genebanks, e.g. the Center for Genetic Resources in the Netherlands (CGN), which alone host more than 1500 L. serriola wild type accessions. Other public genebank where wild type lettuce relatives can be obtained are hosted by the USDA (United States Department of Agriculture) and CGIAR (Consortium of International Agriculture Research Center).

TABLE 2
Markers to track B. lactucae resistance allele on chromosome 9.

| | | SNP Position | | SNP | | | |
|---|---|---|---|---|---|---|---|
| | | in Public | | position in | | | Marker |
| | | Genome | Marker | marker | | Favorable | Sequence |
| Marker | Chr | (bp) | Size | (bp) | SNP | Allele | (SEQ ID NO) |
| M3 | 9 | 176,238,419 | 209 | 106 | T/C | C | 3 |
| M4 | 9 | 177,199,102 | 121 | 61 | A/G | A | 4 |
| M1 | 9 | 181,160,842 | 121 | 61 | A/G | | 1 |
| M5 | 9 | 188,962,368 | 601 | 301 | T/A | A | 5 |
| M6 | 9 | 192,658,443 | 201 | 101 | C/T | C | 6 |
| M2 | 9 | 195,030,172 | 1528 | 352 | A/G | | 2 |
| M7 | 9 | 198,494,489 | 1474 | 194 | C/T | C | 7 |

### Example 3. Testing of Alleles for Resistance to Downy Mildew

The alleles were tested for downy mildew resistance in accordance with the protocol set forth in Example 1. The alleles exhibited resistance for the relevant European isolates including Bl: 17-37EU. It is noted that isolates 1-16 were not commercially relevant in Europe at the time and isolates 17-28 are currently no longer commercially relevant in Europe. The alleles also exhibited resistance for the relevant U.S. isolates including Bl: 5-9US. It is noted that isolates 1-4 are no longer commercially relevant in the U.S.

### Example 4. Testing of Alleles for Resistance to Bremia lactucae Races Bl: 38-40EU

The alleles were tested against additional races of Bremia lactucae, such as those identified and denominated by the International Bremia Evaluation Board. For example, the alleles were tested for resistance to Bremia lactucae races Bl: 38-40EU in accordance with the protocol set forth in Example 1. The results demonstrated that the B. lactucae resistance allele on chromosome 9 found in Lactuca serriola line LAC23B confers resistance to Bremia lactucae races Bl: 38-40EU; the B. lactucae resistance allele on chromosome 9 from Lactuca serriola found in Lactuca sativa line CHD-8W22-6051V confers resistance to Bremia lactucae races Bl: 38EU and Bl: 39EU; and the B. lactucae resistance allele on chromosome 9 from Lactuca serriola found in Lactuca sativa line SVLD0093 confers resistance to Bremia lactucae races Bl: 38EU and Bl: 39EU.

It is noted that new race Bl: 38EU, with IBEB-D sextet code 46-15-38, was first identified in Portugal. Bl: 38EU was found repeatedly, not only in Portugal but also in France, North Spain, South UK, Hungary, Switzerland, and South Germany. Bl: 38 breaks the resistance of S18 (R58 gene), and was found more frequently in Southern Europe. It is noted that new race Bl: 39EU, with IBEB-D sextet code 55-15-33, was first identified in France. Bl: 39EU was found repeatedly, not only in West and Central France, but also in North and West Germany, Switzerland, the Netherlands, South UK, and Portugal. Bl: 39 breaks the resistance of S18 (R58 gene), and was found more frequently in Northern Europe. It is also noted that new race Bl: 40EU, with IBEB-D sextet code 62-31-01, was first identified in Spain. Bl: 40EU was found repeatedly, not only in Spain, but also in France, Germany, the Netherlands, the United Kingdom, Italy, and Norway. Bl: 40 breaks the resistance of S11 (R53 gene). The resistance of S11 has been in the market since 2002, and hardly being attacked by Bremia until the beginning of the outbreak in 2022.

### Example 5. Testing of Alleles for Resistance to Bremia lactucae Race Bl: 41EU

The alleles were also tested against Bremia lactucae race Bl: 41EU, which was recently identified and denominated by the International Bremia Evaluation Board. It is noted that new race Bl: 41, with IBEB-D sextet code 62-31-7, was first identified in France, and was found repeatedly in France, Germany, Switzerland, the Netherlands and the United Kingdom. Bl: 41 breaks the resistance of many resistance genes including R53, R55, and R56. The results demonstrated that the B. lactucae resistance allele on chromosome 9 found in Lactuca serriola line LAC23B confers resistance to Bremia lactucae races Bl: 41EU.

Those of skill in the art will appreciate that new races continue to emerge and/or be newly identified, and that commercially relevant races can change across seasons or growing regions

1. An elite Lactuca sativa plant comprising a recombinant chromosomal segment from Lactuca serriola on chromosome 9, wherein said chromosomal segment is flanked in the genome of said plant by marker locus M1 (SEQ ID NO: 1) and marker locus M2 (SEQ ID NO: 2), and wherein said chromosomal segment comprises an allele conferring resistance to Bremia lactucae relative to a plant lacking said chromosomal segment.

2. The plant of claim 1, wherein said plant further comprises at least one of the following:
a) said chromosomal segment is flanked in the genome of said plant by marker locus M1 (SEQ ID NO: 1) and marker locus M6 (SEQ ID NO: 6)
b) said chromosomal segment comprises a marker locus selected from the group consisting of marker locus M5 (SEQ ID NO: 5) and marker locus M6 (SEQ ID NO: 6) on chromosome 9;
c) said chromosomal segment comprises marker locus M5 (SEQ ID NO: 5) on chromosome 9;
d) the plant is homozygous for said chromosomal segment;
e) a representative sample of seed comprising said allele has been deposited under NCMA Accession No. 202212295, NCMA Accession No. 202307003, or NCMA Accession No. 202306045;
f) said chromosomal segment confers resistance to at least Bremia lactucae races Bl: 17-28EU; of
g) said chromosomal segment confers resistance to at least Bremia lactucae races Bl: 29-37EU;
h) said chromosomal segment confers resistance to at least Bremia lactucae races Bl: 5-9US;
i) said chromosomal segment confers resistance to at least Bremia lactucae races Bl: 38EU and Bl: 39EU;
j) said chromosomal segment confers resistance to at least Bremia lactucae races Bl: 40EU and Bl: 41EU;
k) said chromosomal segment confers resistance to at least Bremia lactucae races Bl: 38-41EU; or
l) said plant comprises a marker locus associated with said chromosomal segment selected from the group consisting of marker locus M3 (SEQ ID NO: 3) and marker locus M4 (SEQ ID NO: 4).

3-7. (canceled)

8. The plant of claim 1 wherein said plant comprises:
a) a marker locus selected from the group consisting of marker locus M3 (SEQ ID NO: 3), marker locus M4 (SEQ ID NO: 4), marker locus M5 (SEQ ID NO: 5), and marker locus M6 (SEQ ID NO: 6);
b) marker locus M3 (SEQ ID NO: 3), M4 (SEQ ID NO: 4), and M6 (SEQ ID NO: 6);
c) marker locus M5 (SEQ ID NO: 5), and M6 (SEQ ID NO: 6);
d) marker locus M3 (SEQ ID NO: 3), and M6 (SEQ ID NO: 6); or
e) marker locus M5 (SEQ ID NO: 5).

9. A seed that produces the plant of claim 1.

10. A plant part of the plant of claim 1 wherein said plant part comprises said chromosomal segment.

11. The plant part of claim 10, wherein said plant part is a cell, a seed, a root, a stem, a leaf, a head, a flower, or pollen.

12. (canceled)

13. A tissue culture comprising the cell of claim 11.

14. A recombinant DNA segment comprising a Bremia lactucae resistance allele from Lactuca serriola, wherein said DNA segment comprises the sequence of marker locus M1 (SEQ ID NO: 1) and marker locus M2 (SEQ ID NO: 2).

15. The recombinant DNA segment of claim 14, wherein:
a) said recombinant DNA segment further comprises a marker locus sequence selected from the group consisting of marker locus M3 (SEQ ID NO: 3), marker locus M4 (SEQ ID NO: 4), marker locus M5 (SEQ ID NO: 5), and marker locus M6 (SEQ ID NO: 6);
b) the recombinant DNA segment is further defined as comprised within a plant, plant part, plant cell, or seed; or
c) a representative sample of seed comprising said recombinant DNA segment has been deposited under NCMA Accession No. 202212295, NCMA Accession No. 202307003, or NCMA Accession No. 202306045;
d) said recombinant DNA segment further comprises:
i) marker locus M3 (SEQ ID NO: 3), M4 (SEQ ID NO: 4), and M6 (SEQ ID NO: 6);
ii) marker locus M5 (SEQ ID NO: 5), and M6 (SEQ ID NO: 6);
iii) marker locus M3 (SEQ ID NO: 3), and M6 (SEQ ID NO: 6); or
iv) marker locus M5 (SEQ ID NO: 5).

16. (canceled)

17. A method for producing an elite Lactuca sativa plant with resistance to Bremia lactucae comprising introgressing into said plant a Bremia lactucae resistance allele from Lactuca serriola within a recombinant chromosomal segment flanked in the genome of said plant by marker locus M1 (SEQ ID NO: 1) and marker locus M2 (SEQ ID NO: 2) on chromosome 9; wherein said Bremia lactucae resistance allele confers to said plant resistance to Bremia lactucae relative to a plant lacking said allele, and wherein said introgressing comprises marker-assisted selection.

18. The method of claim 17, wherein:
a) said introgressing comprises:
i) crossing a plant comprising said recombinant chromosomal segment with itself or with a second Lactuca sativa plant of a different genotype to produce one or more progeny plants; and
ii) selecting a progeny plant comprising said recombinant chromosomal segment; or
b) said introgressing further comprises backcrossing or assaying for said resistance to Bremia lactucae.

19. The method of claim 18, wherein:
a) the progeny plant is an F₂-F₆ progeny plant; or
b) selecting a progeny plant comprises detecting nucleic acids comprising marker locus M1 (SEQ ID NO: 1), marker locus M2 (SEQ ID NO: 2), marker locus M3 (SEQ ID NO: 3), marker locus M4 (SEQ ID NO: 4), marker locus M5 (SEQ ID NO: 5), or marker locus M6 (SEQ ID NO: 6).

20. (canceled)

21. A Lactuca sativa plant obtainable by the method of claim 17.

22. A method of obtaining a seed of a Lactuca sativa plant exhibiting resistance to Bremia lactucae, comprising:
a) crossing the Lactuca sativa plant of claim 1 with itself or with a second Lactuca sativa plant of a different genotype; and
b) obtaining a seed resulting from said crossing that comprises said recombinant chromosomal segment.

23. The method of claim 22, wherein:
a) obtaining said seed comprises detecting a marker locus genetically linked to said chromosomal segment;
b) said seed when grown produces an F₂-F₆ progeny plant;
c) said crossing comprises backcrossing;
d) obtaining the seed comprises detecting nucleic acids comprising marker locus M1 (SEQ ID NO: 1), marker locus M2 (SEQ ID NO: 2), marker locus M3 (SEQ ID NO: 3), marker locus M4 (SEQ ID NO: 4), marker locus M5 (SEQ ID NO: 5), or marker locus M6 (SEQ ID NO: 6); or
e) the method further comprises growing a plant from said seed.

24. (canceled)

25. A method of identifying a Lactuca sativa plant exhibiting resistance to Bremia lactucae, comprising:
a) obtaining a population of progeny plants having a parent comprising resistance to Bremia lactucae;
b) screening said population with at least one nucleic acid marker to detect a polymorphism genetically linked to Bremia lactucae resistance; and
c) identifying in said population at least a first progeny plant comprising a haplotype associated with Bremia lactucae resistance, wherein the haplotype comprises a Bremia lactucae resistance allele from Lactuca serriola at a locus flanked in the genome of said plant by marker locus M1 (SEQ ID NO:1) and marker locus M2 (SEQ ID NO:2) on chromosome 9.

26. The method of claim 25, wherein:
a) identifying said progeny plant comprises:
i) detecting a marker locus within or genetically linked to a chromosomal segment flanked in the genome of said plant by marker locus M1 (SEQ ID NO: 1) and marker locus M2 (SEQ ID NO: 2) on chromosome 9; or
ii) detecting at least one polymorphism at a locus selected from the group consisting of M1 (SEQ ID NO: 1), marker locus M2 (SEQ ID NO: 2), marker locus M3 (SEQ ID NO: 3), marker locus M4 (SEQ ID NO: 4), marker locus M5 (SEQ ID NO: 5), and marker locus M6 (SEQ ID NO: 6); or
iii) detecting at least one polymorphism at marker locus M5 (SEQ ID NO: 5);
b) the haplotype comprises a Bremia lactucae resistance allele from Lactuca serriola at a locus flanked in the genome of said plant by marker locus M1 (SEQ ID NO: 1) and marker locus M6 (SEQ ID NO:6) on chromosome 9;
c) said progeny plant is an F₂-F₆ progeny plant;
d) producing said progeny plant comprises backcrossing;
e) screening said population comprises PCR, single strand conformational polymorphism analysis, denaturing gradient gel electrophoresis, cleavage fragment length polymorphism analysis, TAQMAN assay, and/or DNA sequencing; or
f) a representative sample of seed comprising said Bremia lactucae resistance allele has been deposited under NCMA Accession No. 202212295, NCMA Accession No. 202307003, or NCMA Accession No. 202306045.

27-31. (canceled)

32. A method for identifying a lettuce plant comprising a Bremia lactucae resistance allele from Lactuca serriola, comprising:
a) obtaining nucleic acids from at least a first lettuce plant; and
b) identifying in said nucleic acids the presence of at least a first genetic marker indicative of the presence of a chromosomal segment flanked in the genome of said plant by marker locus M1 (SEQ ID NO:1) and marker locus M2 (SEQ ID NO:2) on chromosome 9;
wherein said chromosomal segment comprises a Bremia lactucae resistance allele conferring to said plant increased resistance to Bremia lactucae compared to a plant not comprising said allele.

33. The method of claim 32, wherein:
said identifying comprises detecting a marker genetically linked to marker locus M1 (SEQ ID NO: 1), marker locus M2 (SEQ ID NO: 2), marker locus M3 (SEQ ID NO: 3), marker locus M4 (SEQ ID NO: 4), marker locus M5 (SEQ ID NO: 5), or marker locus M6 (SEQ ID NO: 6); or
the first genetic marker is indicative of the presence of a chromosomal segment flanked in the genome of said plant by marker locus M1 (SEQ ID NO:1) and marker locus M6 (SEQ ID NO:6) on chromosome 9.

34. (canceled)

35. A method of selecting a lettuce plant or lettuce seed, said method comprising:
a) detecting in a population of lettuce plants or lettuce seeds a lettuce plant or lettuce seed comprising an allele from Lactuca serriola that confers to said plant resistance to Bremia lactucae compared to a plant not comprising said allele; and
b) selecting said lettuce plant or lettuce seed comprising said allele from Lactuca serriola that confers to said plant a resistance to Bremia lactucae.

36. The method of claim 35, wherein:
said allele is located on chromosome 9;
said allele is linked to any marker selected from the group consisting of marker locus M1 (SEQ ID NO: 1), marker locus M2 (SEQ ID NO: 2), marker locus M3 (SEQ ID NO: 3), marker locus M4 (SEQ ID NO: 4), marker locus M5 (SEQ ID NO: 5), or marker locus M6 (SEQ ID NO: 6) on chromosome 9;
said plant is homozygous for said allele from Lactuca serriola;
said plant is heterozygous for said allele from Lactuca serriola; or
said lettuce plant or lettuce seed is an elite plant or a seed of an elite plant.

37-39. (canceled)

40. The method of claim 36, wherein:
said allele is linked to:
a) marker locus M3 (SEQ ID NO: 3), M4 (SEQ ID NO: 4), and M6 (SEQ ID NO: 6);
b) marker locus M5 (SEQ ID NO: 5) and M6 (SEQ ID NO: 6);
c) marker locus M3 (SEQ ID NO: 3) and M6 (SEQ ID NO: 6); or
d) marker locus M5 (SEQ ID NO: 5); or
marker locus M3 comprises a C at position 106 of SEQ ID NO:3;
marker locus M4 comprises an A at position 61 of SEQ ID NO:4;
marker locus M5 comprises an A at position 301 of SEQ ID NO:5; or
marker locus M6 comprises a C at position 101 of SEQ ID NO:6.

41. (canceled)

42. (canceled)

43. A plant identifiable by the method according to claim 35.

44. A nucleic acid molecule comprising an allele conferring to a lettuce plant resistance to Bremia lactucae compared to a plant not comprising said allele, wherein the nucleic acid molecule is obtainable or can be obtained from seed deposited under NCMA Accession No. 202212295, NCMA Accession No. 202307003, or NCMA Accession No. 202306045, and wherein the nucleic acid molecule comprises at least one marker selected from the group consisting of marker locus M3 (SEQ ID NO: 3), marker locus M4 (SEQ ID NO: 4), marker locus M5 (SEQ ID NO: 5), and marker locus M6 (SEQ ID NO: 6).

45. The nucleic acid molecule according to claim 44, wherein:
said nucleic acid molecule comprises:
a) marker locus M3 (SEQ ID NO: 3), M4 (SEQ ID NO: 4), and M6 (SEQ ID NO: 6);
b) marker locus M5 (SEQ ID NO: 5) and M6 (SEQ ID NO: 6);
c) marker locus M3 (SEQ ID NO: 3) and M6 (SEQ ID NO: 6); or
d) marker locus M5 (SEQ ID NO: 5);
the at least one marker locus comprises:
SEQ ID NO:3 comprising a C at position 106;
SEQ ID NO:4 comprising an A at position 61;
SEQ ID NO:5 comprising an A at position 301; or
SEQ ID NO:6 comprising a C at position 101; or
the nucleic acid molecule further comprises at least one marker locus selected from the group consisting of marker locus M1 (SEQ ID NO: 1) and marker locus M2 (SEQ ID NO: 2).

46. (canceled)

47. (canceled)

48. The nucleic acid molecule according to claim 45, wherein the at least one marker locus comprises:
SEQ ID NO:1 comprising an A at position 61;
SEQ ID NO:1 comprising a G at position 61;
SEQ ID NO:2 comprising an A at position 61; or
SEQ ID NO:2 comprising a G at position 61.


SEQUENCE LISTING (7 sequences)

SEQ ID NO: 1 - DNA, 121 bp, Lactuca serriola
  source 1..121: mol_type = genomic DNA
acagaaggttgaaatggatcctccgacggcgttaactctggcgttacctggaacaggttcrtattcgtatgagatctgtgatgaagtgaagaagaagatcaatgcgacatcgtttagcaat

SEQ ID NO: 2 - DNA, 1528 bp, Lactuca serriola
  source 1..1528: mol_type = genomic DNA
aaagacagcacaacgcaaaaanggggggcaaaaagtgatcttccaagatgnagaaaaccctaaaaatggaaaactanaagcagcgagaaggaccancacttcgattcctttctcaatctctgntncnatgnaaatcacattgcatttcaagatctncnctgcgtaaantaaagggnttgngatttggatatggcaaactctaaaggtccatcancaacaananacatggtgtacaatggaagacactctttactcccccctaaatgtccatttccaaccactcctccatcatattctgattacatctcanatcctgcanttggaccaaaaggaataccaaagtacagagatrtcaattcacntcatcaacgtgcttcatctgaaagncttctnattgaanaacaaccttcttggcttgatgagcntcttaatgaaccanaaacacctgtccnaagaggccatanacgttcatcaagtgactctttnacatatctggaagcanccaattctgntaatgtagaatacggngtaaaagntgaacacaanataagaaacttancttctgttccttcatgggggncccacnattttgatttttatanagatgctagaagtgtcccnaataatcctttgatgaatatgaatcctaagtcaagggcatgggattcaccttcaccacanantggtcntcaaagatcggtatcattgggtgcttcaaatgagattcgtgggattgtntcatcaattagggaaaaacaagatggaggtgaatcnggcccacatgattcatctgaagatgcaaaccatgaaagaaaagattcttcagaaactgatacaaaacgtgccaaacagcagtttgctcaaagatcacgggtgaggaagcttcagtatattgctgagctggaaaggaatgtacaggctttacaggcagatggatctgaggtgtcagctgagctggaattcttaaaccagcaaagtcttattttgagcatggagaataaggcactgaaacaacgattggagaatttggctcaagagcaacttattaagtgtttggagcatgaagttttggagagagagctcggaagactacgggccttgtatcaccaccaacaacaaccacaaccaccacaaagccaccaccggagaaccgctagtcgggaaagacttgattcccaatttgcaaacctttctttgaaaaataccgactctacttctgttcataacaaccctgtttctagccaccaacagccgccacgtatctaaaaaataaaaataattattttctaatttcagtggcttgatttggcttttggggtttgtgtttgtgtttgtgtttatgataaaaaaaaatagcataatcttacatgcgcctggtagatgttccctttggaaatcgtaaaatttttctgattaggaactcaccggaggtggaaatttgggggaaatgatataaaaagtttcagtttcccatttggtgttgggtggatagttgttgtcatcatagacttgtttacttaatcatgaa

SEQ ID NO: 3 - DNA, 209 bp, Lactuca serriola
  source 1..209: mol_type = genomic DNA
gaaagtctatttttttggtcatttgaagcttcgatacatccacactgctcaaaatctttgcaacactaatgagttcatttagattcattagagtaaagtcttccaytgagaagtcgaatgccacattgttagcacgaacaacatagtatcgaaagttttgcaacaatccttcaaacaatgtatccttggagatgtttagcactttcaca

SEQ ID NO: 4 - DNA, 121 bp, Lactuca serriola
  source 1..121: mol_type = genomic DNA
aaagaagtaactaatttatttgcatctgatgggataataggagggaaactctcagataaarcgctcagaaaggccttggaaatcgctatatcaccacatctatcattaanagcaataaaaa

SEQ ID NO: 5 - DNA, 601 bp, Lactuca serriola
  source 1..601: mol_type = genomic DNA
tttcatgttttggatggatcagttttctggattggacgattagatactgtttatagcaatcaaatttttttggttagttacttggtttttgtcctttaatatgattagtgtttatcatttataatggtggaatgttttcggttcatactttttgatggactagtggttggttaatagttttaaattttctaacaactacattttagaaaaccaaggcacaagatggaacctaagtacctaacgtctgaggcacataattttataattgaaatttagagtttaaacattggtacattcaaawttcaaaacattgtgttagccacaatctacctatcacatgatgagtccggtttagtatccaaataatgcattttaccttttttcttttaaatttgaaaaaattaacaaaactgcaactcctactcaattctcagttccaaaggtttctagaaccggttccaaagaaaaaaaaaccttttgtgagcacttcatcctttcaagattcgccgattcctagcaatttagccgggtaccgacattatgctcaccttagctcatattatctcctaacacccgatttccaactatacatatacataac

SEQ ID NO: 6 - DNA, 201 bp, Lactuca serriola
  source 1..201: mol_type = genomic DNA
tctttaggtaacactctaggaacattctcaaatggaggaacttcagaatcctaatcaaatcagaacaatataagcatacatctaacaattgtcacttagaytccaaatccataataaatatttcaaattgtactaccttgaatgctctattagcgccatccctcctagcccagtttgatgtcttcctattgttctggttct

SEQ ID NO: 7 - DNA, 1474 bp, Lactuca serriola
  source 1..1474: mol_type = genomic DNA
gacacacactctgtgtagcgatggcgaatcggaggttaaaccctacttctctagcctttctagctctacttctgctcttctcgatcgcttctgctaaagtgntctttgaggagcgcttcgatgatggatgggnaagcaggtgggtaaaatctgactggaagaaagatgagaacatggctggggagtggaattayacttctggtaaatggaatggagatgccaatgataaaggtatccaaaccagtgaagactacaggttttatncaatttcagctgagtaccntnaatncagcaacaaggataaaactttagtcttccagttttctgttangcatgaacagaagctcgattgtggtggtggctacatgaaattgctgagnggngatgttgatcaaaagaaatttggnggtgataccccatacagcatcatgtttggacctgatatttgtgggtatgcaacaangaangntcatgcaattctcacatacaatgnagaaancaaattgatnaagaaagangttccttgtgaaactgatcaactttcacatgtttataccttngtcctncgccctgatgccaccnacaccatctcaattgataatgttgagaaacaaacaggaagtttgtactctgattgggatcttcttccagcaaaacagataaaggaccctgaggccaaaaagcctgaagattgggatgagaaggaattcatccctgaccctgaagacaagaagcctgatggttatgatgacatcccnaangagatttcagaccctgatgcnaanaancccagaggattgggatgatgaggaagatggtgngtggactgttccaactattncaanccctgattacnagggtccatggaaggcaaagaaaatcaagaaccccnactacaaaggaaagtggaaggcacccatgattgacaacccagattttaaagatgacccagatctctatgttttccccaagttgaagtatgttggnattgagttgtggcaggntaaatctggaactctntttgacaatgtcttgatatgtgatgatcctgaatatgccaaacaactagtggaagaaacttgggccaaacaaaaagatgctgaaaaggcagcatttgaagagctagaaaagaaaaaggaggaagaggaatcaaaggatgatccngctgattctgatgctgataatgatgatgctgaaccagaagatgaggaagaggctgatgaaaatgatgttaaggatgagctatagaagaggctttcagcagttagagtttaagattttttttttttctattttacttttaagttgagtttctaggnctttttttnaatagagattataaacaactggaaatgnaatgcagatgagattttctgaaccctgaaattgagaantccattggnctcnattnactacangaattttattggaaactgttgtggcggattggaaccggaattgaatggaatggaat